(19) United States
(12) Patent Application Publication
(10) Pub. No.: US 2012/0114668 A1
(43) Pub. Date: May 10, 2012

(54) METHOD FOR MODULATING ANGIOGENESIS USING FIBROMODULIN

(75) Inventors: Irit Adini, Brookline, MA (US); Robert D'Amato, Lexington, MA (US)

(73) Assignee: CHILDREN'S MEDICAL CENTER CORPORATION, Boston, MA (US)

(21) Appl. No.: 13/290,714

(22) Filed: Nov. 7, 2011

Related U.S. Application Data
(63) Continuation-in-part of application No. PCT/US2010/033724, filed on May 5, 2010.
(60) Provisional application No. 61/176,206, filed on May 7, 2009.

(51) Int. Cl. A61K 39/395; A61P 27/02; A61K 31/7088; A61K 31/7105; A61K 38/02

(52) U.S. Cl. 424/172.1; 514/44 A; 514/20.8; 514/44 R; 514/1.1

(57) ABSTRACT

Described herein are compositions and uses thereof to inhibit or enhance the activity of fibromodulin (FMOD). Such compositions are useful in methods for treatment of age-related macular degeneration involving choroid neovascularization in a subject comprising administering a therapeutically effective amount of a fibromodulin activity inhibitor to the subject.

### CROSS REFERENCE TO RELATED APPLICATIONS

This application is a continuation-in-part of the International Application Ser. No. PCT/US2010/033724, filed on May 5, 2010, which claims benefit under 35 U.S.C. §119(e) of the U.S. Provisional Application No. 61/176,206, filed May 7, 2009, the contents of which are incorporated herein by reference in their entirety.

### FIELD OF THE INVENTION

The present invention relates to the modulation of angiogenesis.

### BACKGROUND

Angiogenesis is the generation of new blood vessels into a tissue or organ. Under normal physiological conditions, humans and animals undergo angiogenesis only in very specific, restricted situations. For example, angiogenesis is normally observed in wound healing, fetal and embryonal development, and formation of the corpus luteum, endometrium and placenta.

Angiogenesis is controlled through a highly regulated system of angiogenic stimulators and inhibitors. The control of angiogenesis has been found to be altered in certain disease states and, in many cases, pathological damage associated with the diseases is related to uncontrolled angiogenesis. Both controlled and uncontrolled angiogenesis are thought to proceed in a similar manner. Endothelial cells and pericytes, surrounded by a basement membrane, form capillary blood vessels. Angiogenesis begins with the erosion of the basement membrane by enzymes released by endothelial cells and leukocytes. Endothelial cells, lining the lumen of blood vessels, then protrude through the basement membrane. Angiogenic stimulants induce the endothelial cells to migrate through the eroded basement membrane. The migrating cells form a “sprout” off the parent blood vessel where the endothelial cells undergo mitosis and proliferate. The endothelial sprouts merge with each other to form capillary loops, creating a new blood vessel.

Persistent, unregulated, abnormal and/or undesired angiogenesis occurs in many disease states, tumor metastases, and abnormal growth by endothelial cells. The diverse pathological disease states in which unregulated angiogenesis is present have been grouped together as angiogenic-dependent or angiogenic-associated diseases.

Fibromodulin (FMOD) is a member of a family of small interstitial proteoglycans that also includes decorin, biglycan and lumican. The proteoglycans bind to other matrix macromolecules and thereby help to stabilize the matrix. (Buckwalter et al., 47 Instr. Course Lect 477-86 (1998)). It is thought that they may influence the function of chondrocytes and bind growth factors. Proteoglycan protein cores are structurally related and consist of a central region of leucine-rich repeats flanked by disulfide-bonded terminal domains. Fibromodulin has up to 4 keratin sulfate chains within its leucine-rich domain. It has a wide tissue distribution and is most abundant in articular cartilage, tendon and ligament. It has been suggested that fibromodulin participates in the assembly of the extracellular matrix due to its ability to interact with type I and type II collagen fibrils and to inhibit fribrillogenesis in vitro.

### SUMMARY OF THE INVENTION

Described herein are methods and compositions useful for inhibiting angiogenesis by modulating the activity of fibromodulin (FMOD). Also provided herein are methods for treating an age-related macular degeneration (AMD) with melanocyte-driven angiogenesis or choroid neovascularization by inhibting the activity of FMOD or inhibiting the expression of FMOD.

Embodiments of the inventions described herein are based, at least in part, on the discovery of a potent pro-angiogenic factor, FMOD, that FMOD is highly expressed in the choroid of the eye, that non-pigmented melanocytes and low pigmented melanocytes secrete excess FMOD, and that anti-FMOD antibody or siRNA-FMOD significantly attenuate the potent pro-angiogenic effect of FMOD.

In one embodiment, provided herein is a method of treatment of age-related macular degeneration involving choroid neovascularization, the method comprising administering a therapeutically effective amount of a fibromodulin activity inhibitor to the subject. In one embodiment, the subject has an eye color of 1-12 on the Martin-Schultz scale.

In one embodiment, provided herein is a method of inhibiting choroid neovascularization in the eye of a subject, the method comprising administering a therapeutically effective amount of a fibromodulin activity inhibitor to the subject. In one embodiment, the subject has an eye color of 1-12 on the Martin-Schultz scale.

In one embodiment, provided herein is a method of inhibiting fibromodulin activity in a subject comprising administering a therapeutically effective amount of a fibromodulin activity inhibitor to the subject. In one embodiment, the subject has an eye color of 1-12 on the Martin-Schultz scale.

In one aspect, the methods described herein relate to a fibromodulin activity inhibitor for use in the treatment of age-related macular degeneration with melanocyte-driven angiogenesis in the choroid of the eye.

In another aspect, the methods described herein relate to a fibromodulin activity inhibitor for use in the treatment of a disorder of abnormal, inappropriate and/or undesired angiogenesis.

In another aspect the methods described herein relate to a method for inhibiting melanocyte-driven endothelial cell growth, proliferation and/or migration, the method comprising contacting a cell with a fibromodulin activity inhibitor.

In another aspect the methods described herein relate to a method for inhibiting endothelial cell growth, proliferation and/or migration, the method comprising contacting a cell with a fibromodulin activity inhibitor.

In one embodiment of this aspect and all other aspects described herein, the cell is selected from the group consisting of a primary cell or a cell of a cell line.

In another embodiment of this aspect and all other aspects described herein, the cell is human.

In another aspect the methods described herein relate to a method of treating age-related macular degeneration with melanocyte-driven angiogenesis or choroid neovascularization, the method comprising administering a therapeutically effective amount of a fibromodulin activity inhibitor to a subject.

In one embodiment of this aspect and all other aspects described herein, the subject has wet age-related macular degeneration.

In another embodiment of this aspect and all other aspects described herein, the fibromodulin activity inhibitor is selected from the group consisting of an antibody, an RNA interference molecule, a small molecule, a peptide and an aptamer.

In another embodiment of this aspect and all other aspects described herein, the fibromodulin activity inhibitor comprises an antibody.

Another aspect described herein relates to a method for inhibiting angiogenesis, the method comprising administering a therapeutically effective amount of a fibromodulin activity inhibitor to a subject in need thereof.

Another aspect described herein relates to a method for inhibiting fibromodulin activity in a subject, the method comprising administering a therapeutically effective amount of a fibromodulin activity inhibitor to a subject in need thereof.

Further described herein is the use of a fibromodulin activity inhibitor in the manufacture of a medicament for the treatment of age-related macular degeneration with melanocyte-driven angiogenesis in the choroid of the eye.

In one embodiment, the subject has low pigmented melanocytes, e.g., in the skin.

Another aspect described herein is a method for promoting endothelial cell proliferation and/or migration, the method comprising: contacting an endothelial cell with an agent that activates fibromodulin activity.

In one embodiment of this aspect and all other aspects described herein, the agent is a fibromodulin polypeptide, or a fragment thereof.

In another embodiment of this aspect and all other aspects described herein, the cell is selected from the group consisting of a primary cell or a cell of a cell line.

In another embodiment of this aspect and all other aspects described herein, the cell is human.

Another aspect described herein relates to a method for promoting angiogenesis in a subject, the method comprising: administering a therapeutically effective amount of an agent that activates fibromodulin activity to a subject in need thereof.

In one embodiment of this aspect and all other aspects described herein, the agent is a fibromodulin polypeptide, or a fragment thereof.

In another embodiment of this aspect and all other aspects described herein, the subject is a mammal.

In another embodiment of this aspect and all other aspects described herein, the mammal is a human.

Another aspect described herein relates to the use of a fibromodulin polypeptide or fragment thereof in the manufacture of a medicament for the treatment of a disorder of impaired angiogenesis.

In one embodiment of this aspect and all other aspects described herein, the disorder is impaired response to wound healing.

In another embodiment of this aspect and all other aspects described herein, the disorder is impaired fertility.

Also described herein is a method for promoting wound healing in a subject, the method comprising: administering an agent that activates fibromodulin activity.

Also described herein is the use of a fibromodulin polypeptide or fragment thereof in the manufacture of a medicament for the treatment of a wound.

Definitions

As used herein, the terms “angiogenesis-related disease” and “angiogenesis-associated disease” are used interchangeably herein and refer to any pathological state that is characterized by or involves uncontrolled or undesired growth of blood vessels. In one embodiment, the “angiogenesis-associated disease” is age-related macular degeneration involving inappropriate angiogenesis in the choroid of the eye, often referred to as choroid neovascularization. Melanocyte-driven angiogenesis in the choroid of the eye is a form of choroid neovascularization.

As used herein, the term “age-related macular degeneration involving melanocyte-driven angiogenesis in the choroid of the eye” refers to age-related macular degeneration occurring in an individual with non-pigmented melanocytes (e.g., in an albino individual) or with melanocytes with low pigmentation. The terms “age-related macular degeneration involving melanocyte-driven angiogenesis in the choroid of the eye” and “age-related macular degeneration with melanocyte-stimulated angiogenesis in the choroid of the eye” are used interchangeably.

As used herein, the term “melanocyte-driven angiogenesis” refers to angiogenesis promoted, induced, or stimulated in vivo at least in part by fibromodulin produced or secreted by melanocytes that are non-pigmented or have low pigmentation. If an anti-FMOD agent decreases or blocks angiogenesis in the eye by at least 5%, the angiogenesis is considered to be “melanocyte-driven angiogenesis.” In other embodiments, if anti-FMOD decreases or blocks angiogenesis by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or even up to at least 100%, the angiogenesis is considered to be “melanocyte-driven angiogenesis.” The terms “melanocyte-driven angiogenesis”, “melanocyte-stimulated angiogenesis”, “melanocyte-induced angiogenesis” and “melanocyte-promoted angiogenesis” are used interchangeably.

As used herein, the term “FMOD-driven angiogenesis” refers to angiogenesis promoted, induced, or stimulated in vivo at least in part by fibromodulin. The term “FMOD-driven angiogenesis”, “FMOD-stimulated angiogenesis” and “FMOD-induced angiogenesis” are used interchangeably.

As used herein, the term “melanocytes with low pigmentation” or “low pigmented melanocytes” refer to skin melanocytes that produce a low level of the pigment melanin. Melanocyte expression of the pigment melanin falls on a broad spectrum in humans, from the relatively low expression seen in those of Caucasian descent, for example, to the relatively high expression in those, for example, of African descent. As used herein, the term “low pigmented melanocyte” refers to a melanocyte in which melanin expression is more than one standard deviation less than the median in a reference population. The term “high pigmented melanocyte” refers to a melanocyte in which melanin expression is more than one standard deviation greater than the median expression in a reference population. The reference population can be the same for the determination of high and low pigmented melanocytes. In one embodiment, the reference population can be a mixed race population found in an American urban city. The reference population can vary from five or more, e.g., 5, 10, 100, 1000, and 5000. The level of melanin can be determined by any methods known in the art, e.g., as described in Rosenthal, et al., Analytical Biochemistry, 1973, vol. 56, pages 91-99, and this reference is expressive incorporated herein by reference in its entirety.

As used herein, the term “less skin pigmentation” with respect to subjects, mammals and humans refers to reduced melanin expression in the skin of more than one standard deviation less than the median in a reference population. The reference level is the average level of melanin expressed in skin (dermal) melanocytes of a population of people as described herein.

As used herein, the term “non-pigmented melanocytes or melanocytes with no pigmentation” refers to melanocytes of an albino individual. An albino individual has melanocytes that are unable to produce melanin for any one of a variety of reasons, including genetic defect in the enzyme tyrosinase. The albino individual can have oculocutaneous albinism (i.e., non-pigmentation in both skin and eye) or ocular albinism, wherein only the eyes lack pigment.

As used herein, the term “choroidal neovascularization” or “choroid neovascularization” is a process in which new blood vessels grow (i.e., neovascularization) in the choroid (the area of the eye containing most blood vessels), through the Bruch membrane and invade the subretinal space.

As used herein, the term “therapeutically effective amount” refers to the amount of an agent that is effective, at dosages and for periods of time necessary to achieve the desired therapeutic result, e.g., a diminishment or prevention of angiogenesis. A therapeutically effective amount of the agents, factors, or inhibitors described herein, or functional derivatives thereof, may vary according to factors such as disease state, age, sex, and weight of the subject, and the ability of the therapeutic compound to elicit a desired response in the subject. The effective amount of a given therapeutic agent will also vary with factors such as the nature of the agent, the route of administration, the size and species of the mammal to receive the therapeutic agent, and the purpose of the administration. A therapeutically effective amount is also one in which any toxic or detrimental effects of the therapeutic agent are outweighed by the therapeutically beneficial effects. The effective amount in each individual case may be determined empirically by a skilled artisan according to established methods in the art and without undue experimentation. In general, an inhibitor is determined to be “therapeutically effective” in the methods described herein if (a) measurable symptom(s) of angiogenesis or an angiogenesis-related disease, (e.g., capillary density, tumor growth, rate of vessel formation) are decreased by at least 10% compared to the measurement prior to treatment onset, (b) the progression of the disease is halted (e.g., patients do not worsen, new vessels do not form, or the tumor does not continue to grow, or (c) symptoms are reduced or even ameliorated, for example, by measuring a reduction in tumor size or a reduction in vessel infiltration in the eye or elsewhere. Efficacy of treatment can be judged by an ordinarily skilled practitioner. Where promotion of angiogenesis is desired, e.g., in promotion of wound healing, an agent, e.g., a FMOD polypeptide is “therapeutically effect” if angiogenesis or one or more markers of angiogenesis or wound healing are increased by at least 10% relative to angiogenesis or the marker measured in the absence of that agent. Efficacy can be assessed in animal models of angiogenesis, cancer and tumor, for example treatment of a rodent with an experimental cancer, and any treatment or administration of the compositions or formulations that leads to a decrease of at least one symptom of the cancer, for example a reduction in the size of the tumor or a cessation or slowing of the rate of growth of the tumor indicates effective treatment. Alternatively, anti-angiogenesis efficacy can be assessed in an animal model of angiogenesis, such as e.g., hindlimb ischemia, wherein a treatment is considered efficacious if there is a reduction in new vessel formation or re-perfusion of the hind limb compared to untreated animals. As yet another alternative, a corneal pocket assay, aortic ring assay or CAM assay can be used to predict treatment efficacy for a given agent.

As used herein, the term “inhibiting angiogenesis” refers to a decrease in a measurable marker of angiogenesis by at least 10%, at least 20%, at least 30%, at least 40%, at least 50%, at least 60%, at least 70%, at least 80%, at least 90%, at least 95%, at least 99%, or even 100% (i.e., absent) in the presence of a fibromodulin activity inhibitor compared to the level of the measurable marker in the absence of an inhibitor. Some non-limiting examples of measurable markers of angiogenesis include capillary density, endothelial cell proliferation, endothelial cell migration, and vessel ingrowth.

As used herein, the terms “increasing angiogenesis” or “promoting angiogenesis” refer to an increase in at least one measurable marker of angiogenesis by at least 10%, at least 20%, at least 30%, at least 40%, at least 50%, at least 60%, at least 70%, at least 80%, at least 90%, at least 95%, at least 99%, at least 1-fold, at least 2-fold, at least 5-fold, at least 10-fold, at least 100-fold, at least 1000-fold or more, in the presence of a pro- angiogenic agent relative to that marker in the absence of such agent.

As used herein, the term “fibromodulin activity” refers to the pro-angiogenic effect of fibromodulin. Fibromodulin angiogenic activity and its inhibition can be assessed by measuring endothelial cell growth and migration in vitro. Endothelial cell growth can be determined, for example, by measuring cell proliferation using an MTS assay commercially available from a variety of companies including RnD Systems, and Promega, among others. Endothelial cell migration can be assessed, for example, by measuring the migration of cells through a porous membrane using a commercially available kit such as BD BioCoat Angiogenesis System or through a Boyden chamber apparatus. As used herein, the term “inhibition of migration” refers to a decrease in the migration of endothelial cells through a porous membrane (e.g., using a commercially available migration assay kit such as BD BioCoat Angiogenesis System) of at least 10% in the presence of a fibromodulin inhibitor, preferably the decrease is at least 20%, at least 30%, at least 40%, at least 50%, at least 60%, at least 70%, at least 80%, at least 90%, at least 95%, at least 99% decrease in the migration of endothelial cells through a porous membrane, or even 100% (i.e., no migration) in the presence of a fibromodulin inhibitor. As used herein, the term “promoting migration” refers to an increase in the migration of endothelial cells through a porous membrane of at least 10% in the presence of an agent that enhances fibromodulin activity, preferably the increase is at least 20%, at least 30%, at least 40%, at least 50%, at least 60%, at least 70%, at least 80%, at least 90%, at least 95%, at least 99%, at least 1-fold, at least 2-fold, at least 5-fold, at least 10-fold, at least 100-fold, at least 1000-fold or more in the presence of an agent that enhances fibromodulin activity, as that term is used herein.

The anti-angiogenic activity of a fibromodulin inhibitor can also be assessed in vivo by a decrease in capillary density or neovascular infiltration using a Matrigel plug assay as described by e.g., Kragh M, et al., (2003) (Kragh M, Hjarnaa P J, Bramm E, Kristjansen P E, Rygaard J, and Binderup L. Int J Oncol. (2003) 22(2):305-11, which is herein incorporated by reference in its entirety) in a mammal treated with a fibromodulin inhibitor, compared to capillary density or neovascular infiltration observed in the absence of a fibromodulin inhibitor. A “decrease in capillary density” means a decrease of at least 5% in the presence of a fibromodulin inhibitor compared to untreated subjects; preferably a decrease in capillary density is at least 10%, at least 20%, at least 30%, at least 40%, at least 50%, at least 60%, at least 70%, at least 80%, at least 90%, at least 95%, at least 99% lower, or even 100% (i.e., absent) in the presence of a fibromodulin inhibitor compared to that measured in the absence of fibromodulin inhibitor administration.

Pro-angiogenic activity of a fibromodulin polypeptide can be measured in similar angiogenesis assay as described herein or known in the art. An “increase in capillary density” means an increase of at least 5% in the presence of an exogenous fibromodulin polypeptide relative to the absence of exogenous fibromodulin polypeptide, preferably at least 10% at least 20%, at least 30%, at least 40%, at least 50%, at least 60%, at least 70%, at least 80%, at least 90%, at least 95%, at least 99%, at least 1-fold, at least 2-fold, at least 5-fold, at least 10-fold, at least 100-fold, at least 1000-fold or more, in the presence of a pro-angiogenic agent relative to that marker in the absence of such agent.

As used herein, the term “inhibiting fibromodulin activity” refers to a decrease in the pro-angiogenic activity of fibromodulin by at least 10%, at least 20%, at least 30%, at least 40%, at least 50%, at least 60%, at least 70%, at least 80%, at least 90%, at least 95%, at least 99%, or even 100% (i.e., no activity) in the presence of a fibromodulin activity inhibitor compared to the pro-angiogenic activity of fibromodulin in the absence of an inhibitor. As used herein, the term “inhibiting endothelial cell proliferation and/or migration” refers to a decrease in the proliferation and/or migration of endothelial cells of at least 10%, at least 20%, at least 30%, at least 40%, at least 50%, at least 60%, at least 70%, at least 80%, at least 90%, at least 95%, at least 99%, or even 100% (i.e., no growth) in the presence of a fibromodulin activity inhibitor compared to the level of proliferation and/or migration in the absence of an inhibitor.

As used herein, the terms “increasing fibromodulin activity” or “promoting fibromodulin activity” refers to an increase in the pro-angiogenic activity of fibromodulin by at least 10% at least 20%, at least 30%, at least 40%, at least 50%, at least 60%, at least 70%, at least 80%, at least 90%, at least 95%, at least 99%, at least 1-fold, at least 2-fold, at least 5-fold, at least 10-fold, at least 100-fold, at least 1000-fold or more, in the presence of a pro-angiogenic agent relative to fibromodulin activity in the absence of such agent.

As used herein, the term “fibromodulin polypeptide” refers to a polypeptide of SEQ ID NO. 1 (Genbank Accession No. NM_{—}002023) or to a conservative substitution variant or fragment thereof that retains fibromodulin activity as that term is defined herein. It should be understood that the carbohydrate moieties of fibromodulin can be involved in fibromodulin pro-angiogenic activity, including, e.g., N-linked keratin sulfate chains. The leucine-rich repeats in the C-terminal domain of the fibromodulin polypeptide have been implicated in the binding of fibromodulin to type I collagen and can play a role in fibromodulin pro-angiogenic activity. See e.g., Kalamajski and Oldberg, (2007) J Biol Chem 282:26740-26745, which highlights the role of leucine-rich repeats in type-I collagen binding. By “retaining fibromodulin activity” is meant that a polypeptide retains at least 50% of the fibromodulin activity of a polypeptide of SEQ ID NO. 1. Also encompassed by the term “fibromodulin polypeptide” are mammalian homologs of human fibromodulin and conservative substitution variants or fragments thereof that retain fibromodulin activity. In one aspect, such homologs or conservative variants thereof stimulate human endothelial cell growth and/or migration as measured, for example, as described herein.

The term “variant” as used herein refers to a polypeptide or nucleic acid that is “substantially similar” to a wild-type fibromodulin polypeptide or polynucleic acid. A molecule is said to be “substantially similar” to another molecule if both molecules have substantially similar structures (i.e., they are at least 50% similar in amino acid sequence as determined by BLASTp alignment set at default parameters) and are substantially similar in at least one relevant function (e.g., effect on cell migration). A variant differs from the naturally occurring polypeptide or nucleic acid by one or more amino acid or nucleic acid deletions, additions, substitutions or side-chain modifications, yet retains one or more specific functions or biological activities of the naturally occurring molecule. Amino acid substitutions include alterations in which an amino acid is replaced with a different naturally-occurring or a non-conventional amino acid residue. Some substitutions can be classified as “conservative,” in which case an amino acid residue contained in a polypeptide is replaced with another naturally occurring amino acid of similar character either in relation to polarity, side chain functionality or size. Substitutions encompassed by variants as described herein can also be “non-conservative,” in which an amino acid residue which is present in a peptide is substituted with an amino acid having different properties (e.g., substituting a charged or hydrophobic amino acid with an uncharged or hydrophilic amino acid), or alternatively, in which a naturally-occurring amino acid is substituted with a non-conventional amino acid. Also encompassed within the term “variant,” when used with reference to a polynucleotide or polypeptide, are variations in primary, secondary, or tertiary structure, as compared to a reference polynucleotide or polypeptide, respectively (e.g., as compared to a wild-type polynucleotide or polypeptide). Polynucleotide changes can result in amino acid substitutions, additions, deletions, fusions and truncations in the polypeptide encoded by the reference sequence. Variants can also include insertions, deletions or substitutions of amino acids, including insertions and substitutions of amino acids and other molecules) that do not normally occur in the peptide sequence that is the basis of the variant, including but not limited to insertion of ornithine which does not normally occur in human proteins.

The term “derivative” as used herein refers to peptides which have been chemically modified, for example by ubiquitination, labeling, pegylation (derivatization with polyethylene glycol) or addition of other molecules. A molecule is also a “derivative” of another molecule when it contains additional chemical moieties not normally a part of the molecule. Such moieties can improve the molecule's solubility, absorption, biological half life, etc. The moieties can alternatively decrease the toxicity of the molecule, or eliminate or attenuate an undesirable side effect of the molecule, etc. Moieties capable of mediating such effects are disclosed in Remington's Pharmaceutical Sciences, 18th edition, A. R. Gennaro, Ed., MackPubl., Easton, Pa. (1990).

The term “functional” when used in conjunction with “derivative” or “variant” refers to polypeptides which possess a biological activity that is substantially similar to a biological activity of the entity or molecule of which it is a derivative or variant. By “substantially similar” in this context is meant that at least 50% of the relevant or desired biological activity of a corresponding wild-type peptide is retained. In the instance of promotion of angiogenesis, for example, an activity retained would be promotion of endothelial cell migration; preferably the variant retains at least 60%, at least 70%, at least 80%, at least 90%, at least 95%, at least 100% or even higher (i.e., the variant or derivative has greater activity than the wild-type), e.g., at least 110%, at least 120%, or more compared to a measurable activity (i.e., promotion or inhibition of endothelial cell migration) of the wild-type polypeptide.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

### BRIEF DESCRIPTION OF THE FIGURES

FIG. 1 shows that fibromodulin (FMOD) increases human microvessel endothelial cell (HMVEC) migration.

FIG. 2 shows that fibromodulin increases human microvessel endothelial cell (HMVEC) proliferation.

FIG. 3A shows that recombinant fibromodulin increases angiogenesis in mouse ear/wound healing model.

FIG. 3B shows the quantification of the recombinant fibromodulin-induced increase in angiogenesis in a mouse ear wound healing model presented as the number of vessels in the ear/wound.

FIG. 4 shows that fibromodulin increases choroidal neovascularization (CNV) in a laser induced CNV mice model.

FIG. 5 shows that fibromodulin secreted from non-pigmented melanocytes induces human microvessel endothelial cells (HMVEC) migration. The histogram shows that the knockdown expression of fibromodulin by siRNA-fibromodulin in non-pigmented melanocytes reduces migration of (HMVEC) exposed to the conditioned medium of non-pigmented melanocytes.

FIG. 6 shows that an anti-fibromodulin antibody decreases endothelial cell migration.

FIG. 7A shows that fibromodulin induces angiogenesis as effectively as human recombinant VEGF 165 in an in vivo corneal micropocket assay.

FIG. 7B shows that an antibody against FMOD inhibit angiogenesis in mice in an in vivo corneal micropocket assay.

FIG. 8A shows increased retinal neovascularization in 4 day old pups injected with recombinant FMOD in the vitreous.

FIG. 8B shows decreased peripheral avascular areas in 4 day old pups injected with recombinant FMOD in the vitreous.

FIG. 8C shows increased neovascularization density in 4 day old pups injected with recombinant FMOD in the vitreous.

FIG. 9 shows that fibromodulin siRNA reduces tumor growth in vivo.

FIG. 10A shows that fibromodulin induces HMVEC migration, proliferation and sprouting from HMVEC coated beads.

FIG. 10B shows that fibromodulin induction of HMVEC sprouting from HMVEC coated beads is comparable to those induced by VEGF.

FIG. 10C shows the percentage of sprouts induced by fibromodulin or VEGF.

FIG. 11 shows that fibromodulin induces angiogenesis in an in vivo corneal micropocket assay as quantified by vessel area, and that the angiogenesis induced is comparable to that of VEGF.

FIG. 12A shows that recombinant (rh) FMOD injected in the vitreous of mice increased CNV progression.

FIG. 12B are representative images of retinal flat-mounts stained with a lectin-FITC from mice injected with rhFMOD; Bars=10 μm.

FIG. 13 shows that fibromodulin and VEGF induces angiogenesis in an in vivo corneal micropocket assay as quantified by vessel length of the new blood vessels.

FIG. 14A shows the relative expression of FMOD mRNA in cultured pigmented and non-pigmented mouse melanocytes determined by real-time RT-PCR analysis.

FIG. 14B shows the relative expression of FMOD protein in cultured pigmented and non-pigmented mouse melanocytes determined by Western Blot analysis.

FIG. 15 shows the localization of FMOD in the mouse retina. FMOD protein is expressed in isolated choroids (the region of the back of the eye with melanocytes) determined by Western Blot analysis.

FIG. 16A shows the migration of endothelial cells induced by conditioned media (CM) from starved pigmented melanocytes, the CM being supplemented with GST as control or with recombinant FMOD.

FIG. 16B shows the migration of endothelial cells induced by conditioned media (CM) from non-starved pigmented melanocytes, the CM being supplemented with GST as control or with recombinant FMOD.

FIG. 16C shows the migration of endothelial cells induced by conditioned media (CM) from non-pigmented melanocytes, the CM being pre-treated with anti-FMOD to remove FMOD from the media.

FIG. 16D shows the migration of endothelial cells induced by conditioned media (CM) from non-pigmented melanocytes that have been treated with siFMOD to knock down the expression of FMOD in the melanocytes.

FIG. 16E shows the Western Blot analysis of FMOD expression in non-pigmented melanocytes that have been treated with siFMOD.

FIG. 17 shows the homozygous knockout of FMOD in Tyrc-2J mice (Albino mice) reduced endothelial cell migration in vivo in a MATRIGEL™ migration assay.

### DETAILED DESCRIPTION OF THE INVENTION

Described herein are methods based, at least in part, on the discovery of strong pro-angiogenic effects of the factor fibromodulin on endothelial cells in vitro and in vivo and the demonstration that fibromodulin inhibitors can inhibit angiogenesis.

The inventors discovered that melanocytes with low pigment, such as in Caucasians, secrete fibromodulin and that the secreted factor has strong pro-angiogenic effects on endothelial cells in vitro and in vivo. The strong pro-angiogenic effects are comparable to those of VEGF. The inventors also discovered that the amount of fibromodulin that is secreted by the melanocyte is inversely correlated with the pigmentation level of the melanocyte. Thus, it was discovered that, in contrast, the pigmented melanocytes in individuals of African descent do not produce and secrete high levels of fibromodulin (samples described herein were obtained from African Americans and are referred to herein by that title). Secreted fibromodulin from non-pigmented melanocytes influences the angiogenic balance in the host tissue containing the melanocytes, e.g., the retina of the eye where there is a concentration of melanocytes. The term “angiogenic balance” refers to the summation of effects of all the local angiogenic stimulators and inhibitors. The inventors further discovered that fibromodulin is highly expressed in the choroid tissue of the retina from albino mice as compared to the choroid tissue of the eye from normal mice.

Thus, for example, described herein are methods for inhibiting melanocyte-driven endothelial cell growth, migration, and/or angiogenesis by administering an inhibitor of a fibromodulin's pro-angiogenic activity. Similarly described are methods of treatment of e.g., diseases or disorders involving or characterized by inappropriate or excess and undesired angiogenesis. Such treatment methods rely upon the administration of an inhibitor of fibromodulin's pro-angiogenic activity. Also described herein are methods based, at least in part, on the discovery that fibromodulin polypeptides can promote angiogenesis, e.g., for wound healing or other situations in which promotion of angiogenesis is desired. Described below are the various elements and considerations necessary for one of skill in the art to practice these and related aspects described and encompassed herein.

Accordingly, in one embodiment, provided herein is a method for inhibiting endothelial cell proliferation and/or migration, the method comprising contacting an endothelial cell with a fibromodulin activity inhibitor. In one embodiment, the endothelial cell proliferation and/or migration are induced at least in part by melanocytes. In one embodiment, the endothelial cell proliferation and/or migration are induced at least in part by fibromodulin. One can determine whether endothelial cell proliferation and/or migration is induced by FMOD, for example, by pre-treatment with an anti-FMOD antibody to remove the FMOD. FMOD-induced endothelial cell proliferation and/or migration would be reduced in the pre-treated condition.

In another embodiment, melanocyte-driven endothelial cell proliferation and/or migration occur in the tissue surrounding melanocytes. In one embodiment, the melanocytes with low pigmentation occur, e.g., in Caucasians. In another embodiment, the endothelial cell proliferation and/or migration occur in the eye of a subject, in the choroid layer of the eye. In another embodiment, the endothelial cell proliferation and/or migration is inappropriate or excess and undesired and thus causes inappropriate or excess and undesired angiogenesis, e.g., as in age-related macular degeneration. In such situations, inhibition of melanocyte-driven endothelial cell growth, migration, and/or angiogenesis can be achieved, for example, by direct injection of the fibromodulin activity inhibitor into the vitreous humor of the affected eye.

Pigmentation in the eye covers a spectrum from none (albino) to very strongly pigmented. As noted above, melanocyte FMOD expression varies approximately inversely with respect to pigment production, and thus also covers a spectrum. Those with relatively light eye color (i.e., low or non-pigmented melanocytes) are more susceptible to AMD. Eye color is quantitated on the “Martin-Schultz scale” discussed herein below. In one embodiment, provided herein is a method of treatment of age-related macular degeneration involving choroid neovascularization in the eye of a subject having an eye color of 1-12 on the Martin-Schultz scale, the method comprising administering a therapeutically effective amount of a fibromodulin activity inhibitor to the subject.

In one embodiment, provided herein is a method of inhibiting choroid neovascularization in the eye of a subject having an eye color of 1-12 on the Martin-Schultz scale, the method comprising administering a therapeutically effective amount of a fibromodulin activity inhibitor to the subject.

In one embodiment, provided herein is a method of inhibiting fibromodulin activity in a subject comprising administering a therapeutically effective amount of a fibromodulin activity inhibitor to the subject. In one embodiment, the subject has an eye color of 1-12 on the Martin-Schultz scale.

The Martin and Schultz scale is a standard color scale commonly used in physical anthropology to establish the eye color of an individual; it was created by the anthropologists Martin and Schultz in the first half of the 20th century. The scale consists of 16 colors (from light blue to dark brown-black) that correspond to the different eye colors observed in nature due to the amount of melanin in the iris (Piquet-Thepot M. -M. —Bulletins et Mémoires de la Société d'anthropologie de Paris, XII° Série, tome 3 fascicule 3, pg. 207,208-(1968), incorporated herein by reference).

The 16 colors of the scale are:
1-2: Blue Iris (1a,1b,1c,2a: Light Blue Iris-2b: Darker Blue Iris)
3: Blue-gray Iris
4: Gray Iris
5: Blue-gray Iris with Yellow/Brown Spots
6: Gray-green Iris with Yellow/Brown Spots
7: Green Iris
8: Green Iris with Yellow/Brown Spots
9-11: Light-brown Iris
10: Hazel Iris
12-13: Medium Brown Iris
14-15-16: Dark-brown and Black Iris

In one embodiment, provided herein is a method of treating an individual for age-related macular degeneration in which the individual is determined to have low pigmented melanocytes in the eye, the method comprising administering a therapeutically effective amount of a fibromodulin activity inhibitor to the individual, wherein if an individual is determined to have high pigmented melanocytes it the eye, the individual is not treated with such an inhibitor.

In one embodiment, the subject determined to have low pigmented melanocytes is a Caucasian or an albino. In another embodiment, the subject determined to have low pigmented melanocytes has less skin pigmentation than a negro, an African-American or an individual of Sub Saharan African descent.

In one embodiment, the subject determined to have low pigmented melanocytes has light eye color, e.g., gray, blue, green, amber or hazel. Various shades of gray, blue, green, amber and hazel are contemplated. In some embodiments, the gray, blue, green, amber and hazel eyes may have small admixture of the pigment melanin.

In one embodiment, provided herein is a method of treatment of age-related macular degeneration with melanocyte-stimulated angiogenesis in the choroid of an eye in a subject, the method comprising administering a therapeutically effective amount of a fibromodulin activity inhibitor to the subject. The inventors discovered that fibromodulin, a pro-angiogenic agent, is highly expressed in the choroid tissue of the eyes in albino mice. Therefore, choroid neovascularization driven by FMOD can lead to age-related macular degeneration.

In one embodiment, the method further comprises selecting a subject diagnosed as having age-related macular degeneration with melanocyte-driven angiogenesis in the choroid of an eye. For example, the individual can be one having non-pigmented melanocytes (e.g., as in an albino) or an individual having low pigmented melanocytes (e.g., as in a Caucasian or fair skinned person as the term is used herein).

In one embodiment, the method further comprises selecting a subject diagnosed as having age-related macular degeneration involving choroid neovascularization.

In one embodiment, the method further comprises selecting a subject diagnosed as having choroid neovascularization.

In one embodiment, the method further comprises selecting a subject having low pigmented melanocytes, such as those having light eye color, e.g., gray, blue, green, amber and hazel. In the embodiments, the gray, blue, green, amber and hazel eyes may have a small admixture of pigment melanin. In one embodiment, the method further comprises selecting a subject having an eye color of 1-12 in the Martin-Schultz scale.

In one embodiment, the subject diagnosed as having age-related macular degeneration has wet macular degeneration. A skilled ophthalmologist would be able to diagnose age-related macular degeneration and whether melanocyte-stimulated angiogenesis in the choroid of an eye is present. Typical examination for AMD includes funduscopic examination, fluorescein angiography, and/or optical coherence tomography. The retinal damage is almost always visible even before symptoms develop. Visual changes can often be detected with an Amsler grid. Fluorescein angiography—a procedure in which a practitioner injects dye into a vein and photographs the retina—is used to confirm the diagnosis of wet AMD. Angiography demonstrates and characterizes subretinal choroidal neovascular membranes and can delineate areas of geographic atrophy.

In one embodiment, the subject is a Caucasian or an albino. In another embodiment, the subject has less skin pigmentation than a negro, an African American or an individual of sub Saharan African descent. In another embodiment, the subject has light eye color, e.g., gray, blue, green, amber and hazel. Various shades of gray, blue, green, amber and hazel are contemplated. In some embodiments, the gray, blue, green, amber and hazel eyes may have a small admixture of the pigment melanin.

In one embodiment, provided herein is a method of inhibiting melanocyte- stimulated angiogenesis in the choroid of an eye in a subject, the method comprising administering a therapeutically effective amount of a fibromodulin activity inhibitor to the subject. In one embodiment, melanocyte-driven angiogenesis refers to the shift in the angiogenic balance toward promoting angiogenesis in tissues where melanocytes are present, e.g., the retina of the eye and the skin, and where there is no or relatively low levels of melanin expressed in these melanocytes, such as in the skin and choroid of Caucasian or albino individuals. In individuals of African descent, the high levels of melanin suppress the production of fibromodulin by melanocytes and thus shift the angiogenic balance towards inhibiting angiogenesis.

In one embodiment, provided herein is a method of inhibiting melanocyte- driven angiogenesis in the choroid of an eye in a subject, the method comprising contacting a melanocyte with a therapeutically effective amount of a fibromodulin activity inhibitor.

In one embodiment, the subject in need of inhibiting melanocyte-driven angiogenesis in the choroid of the eye is a Caucasian or an albino. In another embodiment, the subject has relatively light skin pigmentation. In another embodiment, the subject has light eye color, e.g., gray, blue, green, amber and hazel. Various shades of gray, blue, green, amber and hazel are contemplated. In some embodiments, the gray, blue, green, amber and hazel eyes may have small admixture of pigment melanin. In another embodiment, the subject has an eye color of 1-12 on the Martin-Schultz scale.

In one embodiment, provided herein is a method of inhibiting fibromodulin activity in a subject, the method comprising administering a therapeutically effective amount of a fibromodulin activity inhibitor to the subject. In one embodiment, the subject in need of inhibiting fibromodulin activity is a Caucasian or an albino. In another embodiment, the subject has less skin pigmentation than a negro, an African American or an individual of sub Saharan African descent.

In one embodiment, the fibromodulin activity relates to melanocyte-driven angiogenesis in the eye known as choroid neovascularization.

In one embodiment, the fibromodulin activity occurs in age-related macular degeneration.

In one embodiment, the melanocyte-driven angiogenesis occurs in age-related macular degeneration.

In one embodiment, the fibromodulin activity inhibitor is selected from the group consisting of an antibody, an RNA interference molecule, a small molecule, a peptide and an aptamer.

In one embodiment, the RNA interference molecule is an siRNA. In another embodiment, the RNA interference molecule is a double-stranded RNA (dsRNA). In one embodiment, the dsRNA is an hairpin looped RNA, e.g., a short hairpin looped RNA (shRNA). In one embodiment, the hairpin looped RNA is a shRNA.

In one embodiment, provided herein is a fibromodulin activity inhibitor composition. In one embodiment, the fibromodulin activity inhibitor composition is a pharmaceutical composition comprising a fibromodulin activity inhibitor and a pharmaceutically acceptable carrier.

It is contemplated that a composition comprising at least one RNA interference molecule is used to inhibit the fibromodulin activity, inhibit melanocyte-driven angiogenesis in the choroid of an eye, or treat age-related macular degeneration with melanocyte-driven angiogenesis in the choroid of an eye in a subject. It is also contemplated that the composition comprises two, three, four or more RNA interference molecules targeting different regions of the FMOD coding sequence in order to achieve therapeutic silencing of the FMOD gene. By therapeutic silencing is meant sufficient reduction in FMOD expression to reduce or stop angiogenesis in the affected area in the subject. In one embodiment, the fibromodulin activity inhibitor comprises an antibody targeting the FMOD protein. It is further contemplated that the composition comprises a mixture of one of more antibody, RNA interference molecule, small molecule, peptide and aptamer, all of which targets the FMOD protein, the coding gene or transcript. For example, the composition can comprise two, three, four or more RNA interference molecules and one antibody targeting the FMOD protein.

In one embodiment, the treatment methods further comprising co-administering another therapy for AMD. In one embodiment, the therapy is a conventional therapy that is in practice. Non-limiting examples include intravitreal injection of anti-vascular endothelial growth factor (VEGF) drugs such as ranibizumab, bevacizumab, or pegaptanib; thermal laser photocoagulation of neovascularization; and photodynamic therapy. In addition, corticosteroid drugs (e.g., triamcinolone) can sometimes be injected into the eye to reduce the scaring, and is sometimes injected intraocularly along with an anti-VEGF drug. Other treatments include transpupillary thermotherapy, subretinal surgery, and macular translocation surgery.

In one embodiment, the subject is a mammal. In another embodiment, the subject is a Caucasian or an albino. In another embodiment, the subject has less skin pigmentation.

In one embodiment, the mammal is a human.

In one embodiment, the human is a Caucasian or an albino. In another embodiment, the human has light eye color, e.g., gray, blue, green, amber and hazel. Various shades of gray, blue, green, amber and hazel are contemplated. In some embodiments, the gray, blue, green, amber and hazel eyes may have small admixture of pigment melanin. In another embodiment, the human has an eye color of 1-12 in the Martin-Schultz scale.

In one embodiment, a method is provided for preventing age-related macular degeneration with melanocyte-driven angiogenesis in the choroid of an eye of a subject, the method comprising administering a therapeutically effective amount of a fibromodulin activity inhibitor to the subject. The subject is one that is susceptible or at high risk of developing the condition, e.g., a Caucasian or an albino. In another embodiment, the subject who is susceptible or at high risk of developing the condition has less skin pigmentation.

In one embodiment, the method further comprises selecting a subject who is susceptible or at high risk of developing age-related macular degeneration with melanocyte-stimulated angiogenesis in the choroid of the eye. In one embodiment, the method further comprises selecting a subject who is Caucasian or an albino. Prophylactic administration of a fibromodulin activity inhibitor can help prevent abnormal or undesired angiogenesis in the eye. Prophylactic administration of a fibromodulin activity inhibitor can help prevent choroid neovascularization or limit choroid neovascularization after neovascularization has commenced.

Age-related Macular Degeneration

Age-related macular degeneration (AMD) (also called age-related maculopathy) is a progressive disease of the eye and is associated with choroidal neovascularization. AMD causes progressive damage to the macula, the central and most vital area of the retina, resulting in gradual loss of central vision. It is the most common cause of irreversible loss of central vision in the elderly. It is equally common among men and women.

There are two forms of AMD: dry (atrophic) and wet (neovascular or exudative) AMD. Ninety percent of those with macular degeneration have the dry type. However, 90% of the blindness caused by macular degeneration occurs in the 10% of people who have the wet form.

In dry macular degeneration, the tissues of the macula thin as cells disappear. Both eyes may be affected simultaneously in the dry form. There is no evidence of scarring or of bleeding or other fluid leakage in the macula.

Wet macular degeneration starts off as the dry type. In wet macular degeneration, choroidal neovascularization takes place and results in abnormal blood vessels developing under the macula. The choroid layer of the eye contains most of the eye's blood vessels and is responsible for carrying the nutrients required by the other cellular layers, for example, the retinal pigment epithelium (RPE). The Bruch membrane lays between choriocapillaries of the choroid and the RPE, and acts as a filter between the RPE and choriocapillaries, keeping them in effect separated. Choroidal neovascularization is a process in which new blood vessels grow in the choroid, through the Bruch membrane and invade the subretinal space (hence the description as “wet”). Eventually, a mound of scar tissue develops under the retina. Often times, the wet form develops in one eye first but eventually may affect both eyes.

Fibromodulin

Fibromodulin (FMOD), also called SLRR2E, is a member of a family of small interstitial proteoglycans. The protein is 59 kDa with leucine-rich repeats flanked by disulfide-bonded terminal domains, possessing up to 4 keratan sulfate chains (Takahashi, T., Cho, H. I., Kublin, C. L. & Cintron, C. (1993) J Histochem Cytochem 41:1447-57). Fibromodulin exhibits a wide tissue distribution with the highest concentration found in articular cartilage, tendon, and ligament. The subcellular location of fibromodulin is within the cytosolic proteins with a secretory sequence but no transmembrane or extracellular domain.

While it is not wished to indicate that such activity is critical to the pro-angiogenic activity of fibromodulin, several activities of fibromodulin are worth noting here. A characteristic feature of this protein is its participation in the assembly of the extracellular matrix by virtue of its ability to interact with type I, type II and XII collagen fibrils to form collagen fibrils network (Hedbom, E. & Heinegard, D. (1993) J Biol Chem 268: 27307-12; Font, B., Eichenberger, D., Goldschmidt, D., Boutillon, M. M. & Hulmes, D. J. (1998) Eur J Biochem 254:580-7) and to inhibit fibrillogenesis in vitro (Antonsson, P., Heinegard, D. & Oldberg, A.(1991) J Biol Chem 266:16859-61; Hedlund, H., Mengarelli-Widholm, S., Heinegard, D., Reinholt, F. P. & Svensson, O. (1994) Matrix Biol 14:227-32; Ezura, Y., Chakravarti, S., Oldberg, A., Chervoneva, I. & Birk, D. E. (2000) J Cell Biol 151:779-88; Gori, F., Schipani, E. & Demay, M. B. (2001) J Cell Biochem 82:46-57; Ameye, L. et al. (2002) Faseb J 16: 673-80; Ameye, L. & Young, M. F. (2002) Glycobiology 12:107R-16R; Chakravarti, S. (2002) Glycoconj J 19:287-93) FMOD interaction with transforming growth factor (TGF)-β, a key profibrotic cytokine, is considered to enhance the retention of this growth factor within the ECM, thus regulating TGF-β local action (Burton-Wurster, N. et al. (2003) Osteoarthritis Cartilage 11:167-76; San Martin, S. et al. (2003) Reproduction 125:585-95; Fukushima, D., Butzow, R., Hildebrand, A. & Ruoslahti, E. (1993) J Biol Chem 268:22710-5; Hildebrand, A. et al. (1994) Biochem J 302 (Pt 2):527-34). The protein is involved in a variety of adhesion processes of connective tissue, and with immunoglobulins activating both the classical and the alternative pathways of complement. Further studies revealed that fibromodulin binds directly to the globular heads of Clq, leading to activation of C1. Fibromodulin also binds complement inhibitor factor H (Sjoberg, A. P. et al. (2007) J Biol Chem 282:10894-900; Sjoberg, A., Onnerfjord, P., Morgelin, M., Heinegard, D. & Blom, A. M. (2005) J Biol Chem 280:32301-8)

The fibromodulin gene has been found to be an overexpressed gene in B-cell chronic lymphocytic leukemia and chronic lymphocytic leukemia (CLL). It may serve as a potential tumor-associated antigen (TAA) in CLL (Mayr, C. et al. (2005) Blood 105:1566-73; Mayr, C. et al.(2005) Blood 106:3223-6). The amino acid sequences of human and bovine, rat and murine fibromodulin show an overall homology of 90%, allowing for close translation between human and murine experimental models (Antons son, P., Heinegard, D. & Oldberg, A. (1993) Biochim Biophys Acta 1174:204-6).

Fibromodulin Activity Inhibitors

Essentially any agent that inhibits fibromodulin activity, as that term is defined herein, can be used with the methods described herein. It is preferred, however, that an inhibitor of fibromodulin activity is specific, or substantially specific, for fibromodulin activity inhibition. Further, it is noted that fibromodulin activity can be inhibited by agents that specifically inhibit the expression of the fibromodulin proteoglycan as well as by agents that specifically either bind to, or cleave, the fibromodulin proteoglycan molecule. Some non-limiting examples of agents include antibodies, small molecules, RNA interference molecules, aptamers, ligands, peptides, nucleic acids, or a combination thereof. In addition, expression of a dominant negative mutant of a fibromodulin polypeptide can also be used to inhibit fibromodulin activity. Competitive mutants and/or competitive peptides of a fibromodulin polypeptide are also contemplated for use herein for inhibiting fibromodulin activity.

Inhibitors of fibromodulin can be screened for efficacy by measuring fibromodulin's pro-angiogenic activity in the presence and absence of the inhibitor, using for example, a fibromodulin activity assay performed using methods e.g., as described in the Examples herein. To avoid doubt, an agent that inhibits fibromodulin activity will, at a minimum, reduce the pro-angiogenic activity of fibromodulin, as that term is used herein.

Small Molecule Inhibitors

As used herein, the term “small molecule” refers to a chemical agent including, but not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Small molecules inhibitors of fibromodulin activity can be identified from within a small molecule library, which can be obtained from commercial sources such as AMRI (Albany, N.Y.), AsisChem Inc. (Cambridge, Mass.), TimTec (Newark, Del.), among others, or from libraries as known in the art.

Aptamers

Aptamers are relatively short RNA or DNA oligonucleotides, which bind ligands and are isolated in vitro using, for example, the selection procedure known as SELEX (systematic evolution of ligands by exponential enrichment) (Tuerk & Gold, 1990; Ellington & Szostak, 1990, U.S. Pat. Nos. 5,475,096 and 5,270,163, which are incorporated herein by reference in their entirety). Because the selection procedure is driven by binding of ligands, aptamers bind their ligands with high affinity and fold into secondary structures which are optimized for ligand binding (Herman & Patel, 2000, incorporated herein by reference in its entirety). In this respect aptamers resemble antibodies by selectively binding corresponding ligand from complex chemical or biological mixtures.

The aptamer oligonucleotide of such an embodiment can be any useful aptamer now known or later developed. Methods to design and synthesize aptamers and aptamer binding sequences are known to those of skill in the art.

It is contemplated herein that aptamers directed at binding fibromodulin and inhibiting its pro-angiogenic activity can be used in the methods described herein. The aptamers can be delivered by any method known in the art, e.g., systemic or localized administration, e.g., by local injection or implantation of a dosage form. Direct injection of aptamers to the vitreous humor of the eye is contemplated.

Antibodies

Antibodies can be used to inhibit fibromodulin by e.g., recognition of an epitope such that a bound antibody inhibits fibrodulin's pro-angiogenic activity. Production of antibodies useful for the methods described herein is known to those of skill in the art.

The production of non-human monoclonal antibodies, e.g., murine or rat, can be accomplished by, for example, immunizing the animal with a desired target peptide or polypeptide and preparing hybridomas of spleen cells from the immunized animals, according to well established methods (e.g., See Harlow & Lane, Antibodies, A Laboratory Manual (CSHP NY, 1988, which is herein incorporated by reference in its entirety). Immunogen can be obtained from a natural source, by peptide synthesis or by recombinant expression. Humanized forms of mouse antibodies (e.g., as produced by a hybridoma) can be generated by cloning and linking the CDR regions of the murine antibodies to human constant regions by recombinant DNA techniques. See Queen et al., Proc. Natl. Acad. Sci. USA 86, 10029-10033 (1989) and WO 90/07861 (incorporated by reference herein in their entirety). Human antibodies can be obtained using phage-display methods. See, e.g., Dower et al., WO 91/17271; McCafferty et al., WO 92/01047, which are incorporated herein by reference in their entirety. In these methods, libraries of phage are produced in which members display different antibodies on their outer surfaces. Antibodies are usually displayed as Fv or Fab fragments. Phage displaying antibodies with a desired specificity are selected by binding to a fibromodulin polypeptide or fragments thereof. Increased affinity can be selected by successive rounds of affinity enrichment by binding to the same fragment. Human antibodies against fibromodulin can also be produced from non-human transgenic mammals having transgenes encoding at least a segment of the human immunoglobulin locus and an inactivated endogenous immunoglobulin locus. See, e.g., Lonberg et al., WO93/12227 (1993); Kucherlapati, WO 91/10741 (1991) (each of which is incorporated by reference in its entirety). Human antibodies can be selected by competitive binding experiments, or otherwise, to have the same epitope specificity as a particular mouse antibody. Such antibodies are particularly likely to share the useful functional properties of the mouse antibodies. Human polyclonal antibodies can also be provided in the form of serum from humans immunized with an immunogenic agent. Optionally, such polyclonal antibodies can be concentrated by affinity purification using fibromodulin as an affinity reagent. Human or humanized antibodies can be designed to have IgG, IgD, IgA and IgE constant region, and any isotype, including IgG1, IgG2, IgG3 and IgG4. Antibodies can be expressed as tetramers containing two light and two heavy chains, as separate heavy chains, light chains, as Fab, Fab′F(ab′)2, and Fv, or as single chain antibodies in which heavy and light chain variable domains are linked through a spacer.

Fibromodulin antibodies can be obtained from commercial sources such as e.g., Santa Cruz Biotechnology Inc. (Santa Cruz, Calif.), Millipore (Billerica, Mass.), Novus Biologicals (Littleton, Colo.), AbNova Corporation (Walnut, Calif.), and AbCam (Cambridge, Mass.), among others. A mouse monoclonal antibody against human fibromodulin can be obtained from Acris Antibodies (Herford, Germany), which is distributed in the U.S. by Novus Biologicals (Littleton, Colo.). In addition, the genes encoding, for example, murine or goat anti fibromodulin antibodies provide candidates for humanization for therapeutic purposes. The anti-FMOD antibodies can be delivered by any method known in the art, e.g., systemic or localized administration, e.g., by local injection or implantation of a dosage form. Direct injection of FMOD-directed antibodies to the vitreous humor of the eye is specifically contemplated.

RNA Interference

RNA interference agents can be used with the methods described herein, to inhibit the expression and/or activity of a fibromodulin polypeptide. “RNA interference (RNAi)” is an evolutionarily conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B., J. of Virology 76(18):9225 (2002), herein incorporated by reference in its entirety), thereby inhibiting expression of the target gene. As used herein, “inhibition of target gene expression” includes any decrease in expression or protein activity or level of the target gene or protein encoded by the target gene as compared to a situation wherein no RNA interference has been induced. The decrease can be of at least 20%, 30%, 40%, 50%, 60%, 70%, 80%, 90%, 95% or 99% or more as compared to the expression of a target gene or the activity or level of the protein encoded by a target gene which has not been targeted by an RNA interfering agent. RNA interfering agents contemplated for use with the methods described herein include, but are not limited to, siRNA, shRNA, miRNA, and dsRNAi.

The target gene or sequence of the RNA interfering agent can be a cellular gene or genomic sequence. An siRNA can be substantially homologous to the target gene or genomic sequence, or a fragment thereof. As used in this context, the term “homologous” is defined as being substantially identical, sufficiently complementary, or similar to the target mRNA, or a fragment thereof, to effect RNA interference of the target. Preferably, the siRNA is identical in sequence to its target and targets only one sequence. Each of the RNA interfering agents, such as siRNAs, can be screened for potential off-target effects by, for example, expression profiling. Such methods are known to one skilled in the art and are described, for example, in Jackson et al., Nature Biotechnology 6:635-637 (2003), herein incorporated by reference in its entirety.

It is well within the ability of one skilled in the art to design and test for siRNAs that are useful for inhibiting fibromodulin expression and/or activity. It is important to note that double-stranded siRNA or shRNA molecules that are cleaved by Dicer in the cell can be up to 100 times more potent than a 21-mer siRNA or shRNA molecule supplied exogenously (Kim, D H., et al (2005) Nature Biotechnology 23(2):222-226). Thus, an RNAi molecule can be designed to be more effective by providing a sequence for Dicer cleavage. Methods for effective siRNA design for use in vivo can be found in U.S. Pat. No. 7,427,605, which is herein incorporated by reference in its entirety.

Commercially available RNA interference molecules that target fibromodulin can be obtained from e.g., Santa Cruz Biotechnology Inc. (Santa Cruz, Calif.), Cell Signaling Technologies (Danvers, Mass.), Sigma-Aldrich (St. Louis, Mo.), and Dharmacon Inc.(Lafayette, Colo.), among others.

In Vivo Delivery of RNA Interference (RNAi) Molecules

In general, any method of delivering a nucleic acid molecule can be adapted for use with an RNAi interference molecule (see e.g., Akhtar S. and Julian R L. (1992) Trends Cell. Biol. 2(5):139-144; WO94/02595, which are incorporated herein by reference in their entirety). However, there are three factors that are important to consider in order to successfully deliver an RNAi molecule in vivo: (a) biological stability of the RNAi molecule, (2) preventing non-specific effects, and (3) accumulation of the RNAi molecule in the target tissue. The non-specific effects of an RNAi molecule can be minimized by local administration by e.g., direct injection into a tissue including, for example, a tumor or topically administering the molecule.

Local administration of an RNAi molecule to a treatment site limits the exposure of the e.g., siRNA to systemic tissues and permits a lower dose of the RNAi molecule to be administered. Several studies have shown successful knockdown of gene products when an RNAi molecule is administered locally. For example, intraocular delivery of a VEGF siRNA by intravitreal injection in cynomolgus monkeys (Tolentino, M J., et al (2004) Retina 24:132-138) and subretinal injections in mice (Reich, S J., et al (2003) Mol. Vis. 9:210-216) were both shown to prevent neovascularization in an experimental model of age-related macular degeneration. In addition, direct intratumoral injection of an siRNA in mice reduces tumor volume (Pille, J., et al (2005) Mol. Ther.11:267-274) and can prolong survival of tumor-bearing mice (Kim, W J., et al (2006) Mol. Ther. 14:343-350; Li, S., et al (2007) Mol. Ther. 15:515-523). RNA interference has also shown success with local delivery to the CNS by direct injection (Dorn, G., et al. (2004) Nucleic Acids 32:e49; Tan, P H., et al (2005) Gene Ther. 12:59-66; Makimura, H., et al (2002) BMC Neurosci. 3:18; Shishkina, G T., et al (2004) Neuroscience 129:521-528; Thakker, E R., et al (2004) Proc. Natl. Acad. Sci. U.S.A. 101:17270-17275; Akaneya, Y., et al (2005) J. Neurophysiol. 93:594-602) and to the lungs by intranasal administration (Howard, K A., et al (2006) Mol. Ther. 14:476-484; Zhang, X., et al (2004) J. Biol. Chem. 279:10677-10684; Bitko, V., et al (2005) Nat. Med. 11:50-55).

For administering an RNAi molecule systemically for the treatment of a disease, the RNAi molecule can be either be modified or alternatively delivered using a drug delivery system; both methods act to prevent the rapid degradation of the RNAi molecule by endo- and exo-nucleases in vivo. Modification of the RNAi molecule or the pharmaceutical carrier can also permit targeting of the RNAi molecule to the target tissue and avoid undesirable off-target effects.

RNA interference molecules can be modified by chemical conjugation to lipophilic groups such as cholesterol to enhance cellular uptake and prevent degradation. For example, an siRNA directed against ApoB conjugated to a lipophilic cholesterol moiety was injected systemically into mice and resulted in knockdown of apoB mRNA in both the liver and jejunum (Soutschek, J., et al (2004) Nature 432:173-178). Conjugation of an RNAi molecule to an aptamer has been shown to inhibit tumor growth and mediate tumor regression in a mouse model of prostate cancer (McNamara, J O., et al (2006) Nat. Biotechnol. 24:1005-1015).

In an alternative embodiment, the RNAi molecules can be delivered using drug delivery systems such as e.g., a nanoparticle, a dendrimer, a polymer, liposomes, or a cationic delivery system. Positively charged cationic delivery systems facilitate binding of an RNA interference molecule (negatively charged) and also enhance interactions at the negatively charged cell membrane to permit efficient uptake of an siRNA by the cell. Cationic lipids, dendrimers, or polymers can either be bound to an RNA interference molecule, or induced to form a vesicle or micelle (see e.g., Kim S H., et al (2008) Journal of Controlled Release 129(2):107-116) that encases an RNAi molecule. The formation of vesicles or micelles further prevents degradation of the RNAi molecule when administered systemically. Methods for making and administering cationic-RNAi complexes are well within the abilities of one skilled in the art (see e.g., Sorensen, D R., et al (2003) J. Mol. Biol 327:761-766; Verma, U N., et al (2003) Clin. Cancer Res. 9:1291-1300; Arnold, A S et al (2007) J. Hypertens. 25:197-205, which are incorporated herein by reference in their entirety).

Some non-limiting examples of drug delivery systems useful for systemic administration of RNAi include DOTAP (Sorensen, D R., et al (2003), supra; Verma, U N., et al (2003), supra), Oligofectamine, “solid nucleic acid lipid particles” (Zimmermann, T S., et al (2006) Nature 441:111-114), cardiolipin (Chien, P Y., et al (2005) Cancer Gene Ther. 12:321-328; Pal, A., et al (2005) Int J. Oncol. 26:1087-1091), polyethyleneimine (Bonnet M E., et al (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A., et al (2007) Biochem. Soc. Trans. 35:61-67; Yoo, H., et al (1999) Pharm. Res. 16:1799-1804). In some embodiments, an RNAi molecule forms a complex with cyclodextrin for systemic administration. Methods for administration and pharmaceutical compositions of RNAi molecules and cyclodextrins can be found in U.S. Pat. No. 7,427,605, which is herein incorporated by reference in its entirety. Specific methods for administering an RNAi molecule for the inhibition of angiogenesis can be found in e.g., U.S. Patent Application No. 20080152654, which is herein incorporated by reference in its entirety.

Fibromodulin Polypeptides

A fibromodulin polypeptide or a portion thereof functional to promote angiogenesis can be administered to an individual in need thereof. In one approach, a soluble fibromodulin polypeptide, produced, for example, in cultured cells bearing a recombinant fibromodulin expression vector can be administered to the individual. The fibromodulin polypeptide or portion thereof will generally be administered intravenously. This approach rapidly delivers the protein throughout the system and maximizes the chance that the protein is intact when delivered. Alternatively, other routes of therapeutic protein administration are contemplated, such as by inhalation. Technologies for the administration of agents, including protein agents, as aerosols are well known and continue to advance. Alternatively, the polypeptide agent can be formulated for topical delivery, including, for example, preparation in liposomes. Further contemplated are, for example, transdermal administration, and rectal or vaginal administration. Further options for the delivery of fibromodulin polypeptides as described herein are discussed in the section “Pharmaceutical Compositions” herein below.

Vectors for transduction of a fibromodulin-encoding sequence are well known in the art. While overexpression using a strong non-specific promoter, such as a CMV promoter, can be used, it can be helpful to include a tissue- or cell-type-specific promoter on the expression construct—for example, the use of a skeletal muscle-specific promoter or other cell-type-specific promoter can be advantageous, depending upon what cell type is used as a host. Further, treatment can include the administration of viral vectors that drive the expression of fibromodulin polypeptides in infected host cells. Viral vectors are well known to those skilled in the art.

These vectors are readily adapted for use in the methods of the present invention. By the appropriate manipulation using recombinant DNA/molecular biology techniques to insert an operatively linked fibromodulin encoding nucleic acid segment into the selected expression/delivery vector, many equivalent vectors for the practice of the methods described herein can be generated. It will be appreciated by those of skill in the art that cloned genes readily can be manipulated to alter the amino acid sequence of a protein.

The cloned gene for fibromodulin can be manipulated by a variety of well known techniques for in vitro mutagenesis, among others, to produce variants of the naturally occurring human protein, herein referred to as muteins or variants or mutants of fibromodulin, which may be used in accordance with the methods and compositions described herein. The variation in primary structure of muteins of fibromodulin useful in the invention, for instance, may include deletions, additions and substitutions. The substitutions may be conservative or non-conservative. The differences between the natural protein and the mutein generally conserve desired properties, mitigate or eliminate undesired properties and add desired or new properties. The fibromodulin polypeptide can also be a fusion polypeptide, fused, for example, to a polypeptide that targets the product to a desired location, or, for example, a tag that facilitates its purification, if so desired. Fusion to a polypeptide sequence that increases the stability of the fibromodulin polypeptide is also contemplated. For example, fusion to a serum protein, e.g., serum albumin, can increase the circulating half-life of a fibromodulin polypeptide. Tags and fusion partners can be designed to be cleavable, if so desired. Another modification specifically contemplated is attachment, e.g., covalent attachment, to a polymer. In one aspect, polymers such as polyethylene glycol (PEG) or methoxypolyethylene glycol (mPEG) can increase the in vivo half-life of proteins to which they are conjugated. Methods of PEGylation of polypeptide agents are well known to those skilled in the art, as are considerations of, for example, how large a PEG polymer to use. In another aspect, biodegradable or absorbable polymers can provide extended, often localized, release of polypeptide agents. Such synthetic bioabsorbable, biocompatible polymers, which may release proteins over several weeks or months can include, for example, poly-α-hydroxy acids (e.g. polylactides, polyglycolides and their copolymers), polyanhydrides, polyorthoesters, segmented block copolymers of polyethylene glycol and polybutylene terephtalate (Polyactive™), tyrosine derivative polymers or poly(ester-amides). Suitable bioabsorbable polymers to be used in manufacturing of drug delivery materials and implants are discussed e.g. in U.S. Pat. Nos. 4,968,317; 5,618,563, among others, and in “Biomedical Polymers” edited by S. W. Shalaby, Carl Hanser Verlag, Munich, Vienna, New York, 1994 and in many references cited in the above publications. The particular bioabsorbable polymer that should be selected will depend upon the particular patient that is being treated.

Diseases

Essentially any angiogenesis-associated disease can be treated with the methods and compositions described herein. In one embodiment, methods of treatment described herein include the step of diagnosing an individual with an angiogenesis-associated or angiogenesis-related disease or disorder. While it is anticipated that fibromodulin inhibitors would target angiogenesis in tissues with elevated levels of fibromodulin, such elevated levels are not necessarily required for anti-fibromodulin therapy to be effective.

One example of a disease mediated by angiogenesis is ocular neovascular disease. This disease is characterized by invasion of new blood vessels into the structures of the eye, such as the retina or cornea. It is the most common cause of blindness and is involved in approximately twenty eye diseases. In age-associated macular degeneration, the associated visual problems are caused by an ingrowth of choroidal capillaries through defects in Bruch's membrane with proliferation of fibrovascular tissue beneath the retinal pigment epithelium.

Angiogenic damage is also associated with diabetic retinopathy, retinopathy of prematurity/retrolental fibroplasia, corneal graft rejection and neovascular glaucoma. Other diseases or conditions associated with corneal neovascularization include, but are not limited to, epidemic keratoconjunctivitis, Vitamin A deficiency, trachoma, contact lens overwear, atopic keratitis, superior limbic keratitis, pterygium keratitis sicca, Sjogrens disease, acne rosacea, phylectenulosis, syphilis, Mycobacteria infections, lipid degeneration, chemical burns, bacterial ulcers, fungal ulcers, Herpes simplex infection, Herpes simplex keratitis, Herpes zoster infections, protozoan infections, Kaposi's sarcoma, Mooren's ulcer, Terrien's marginal degeneration, marginal keratolysis, rheumatoid arthritis, systemic lupus, polyarteritis, trauma, Wegener's sarcoidosis, scleritis, Stevens-Johnson's disease, pemphigoid, trachoma and radial keratotomy.

Diseases associated with retinal/choroidal neovascularization include, but are not limited to, diabetic retinopathy, macular degeneration, sickle cell anemia, sarcoidosis, syphilis, pseudoxanthoma elasticum, Paget's disease, vein occlusion, artery occlusion, carotid obstructive disease, chronic uveitis/vitritis, Mycobacteria infections, Lyme disease, systemic lupus erythematosis, retinopathy of prematurity, Eales'disease, Behcet's disease, infections causing retinitis or choroiditis, presumed ocular histoplasmosis, Best's disease, myopia, optic pits, Stargardt's disease, pars planitis, chronic retinal detachment, hyperviscosity syndromes, toxoplasmosis, histoplasmosis, trauma and post-laser complications. Other eye-associated diseases that can involve inappropriate angiogenesis include, but are not limited to, diseases associated with rubeosis (neovascularization of the angle) and diseases caused by the abnormal proliferation of fibrovascular or fibrous tissue, including all forms of prolific vitreoretinopathy.

Another angiogenesis associated disease is rheumatoid arthritis. The blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis.

Angiogenesis may also play a role in osteoarthritis and gout. The activation of the chondrocytes by angiogenic-associated factors contributes to the destruction of the joint. At a later stage, the angiogenic factors promote new bone growth. Therapeutic intervention that prevents the bone destruction could halt the progress of the disease and provide relief for persons suffering with arthritis.

Angiogenesis has been associated with a number of different types of cancer, including solid tumors and blood-borne tumors. Solid tumors with which angiogenesis has been associated include, but are not limited to cancer of the prostate, lung, breast, brain, ovarian, stomach, pancreas, larynx, esophagus, testes, liver, parotid, biliary tract, colon, rectum, cervix, uterus, endometrium, kidney, bladder and thyroid; as well as rhabdomyosarcomas, retinoblastoma, Ewing's sarcoma, neuroblastoma, and osteosarcoma, to name but a few. Tumors in which angiogenesis is important include benign tumors such as acoustic neuroma, neurofibroma, trachoma, and pyogenic granulomas. Prevention of angiogenesis could halt the growth of these tumors and the resultant damage to the animal due to the presence of the tumor.

Angiogenesis is also associated with blood-borne tumors, such as leukemias, any of various acute or chronic neoplastic diseases of the bone marrow in which unrestrained proliferation of white blood cells occurs, usually accompanied by anemia, impaired blood clotting, and enlargement of the lymph nodes, liver and spleen. It is believed to that angiogenesis plays a role in the abnormalities in the bone marrow and lymph nodes that give rise to lymphoma, myelodysplastic syndrome and multiple myeloma.

One of the most frequent angiogenic diseases of childhood is the hemangioma. A hemangioma is a tumor composed of newly-formed blood vessels. In most cases the tumors are benign and regress without intervention. In more severe cases, the tumors progress to large cavernous and infiltrative forms and create clinical complications. Systemic forms of hemangiomas, hemangiomatoses, have a high mortality rate. Therapy-resistant hemangiomas exist that cannot be treated with therapeutics currently in use.

Angiogenesis is prominent in solid tumor formation and metastasis. Angiogenic factors have been found associated with several solid tumors such as rhabdomyosarcomas, retinoblastoma, Ewing's sarcoma, neuroblastoma, and osteosarcoma. A tumor cannot expand without a blood supply to provide nutrients and remove cellular wastes. Tumors in which angiogenesis is important include solid tumors, and benign tumors such as acoustic neuroma, neurofibroma, trachoma and pyogenic granulomas. Prevention of angiogenesis could halt the growth of these tumors and the resultant damage to the animal due to the presence of the tumor.

Angiogenesis is important in two stages of tumor metastasis. The first stage where angiogenesis stimulation is important is in the vascularization of the tumor which allows tumor cells to enter the blood stream and to circulate throughout the body. After the tumor cells have left the primary site, and have settled into the secondary, metastasis site, angiogenesis must occur before the new tumor can grow and expand. Therefore, prevention of angiogenesis could lead to the prevention of metastasis of tumors and possibly contain the neoplastic growth at the primary site.

Knowledge of the role of angiogenesis in the maintenance and metastasis of tumors has led to a prognostic indicator for breast cancer. The amount of neovascularization found in the primary tumor was determined by counting the microvessel density in the area of the most intense neovascularization in invasive breast carcinoma. A high level of microvessel density was found to correlate with tumor recurrence. Control of angiogenesis by therapeutic means can lead to cessation of the recurrence of the tumors.

Angiogenesis is also responsible for damage found in hereditary diseases such as Osler-Weber-Rendu disease, or hereditary hemorrhagic telangiectasia. This is an inherited disease characterized by multiple small angiomas, tumors of blood or lymph vessels. The angiomas are found in the skin and mucous membranes, often accompanied by epitaxis (nose bleeds) or gastrointestinal bleeding and sometimes with pulmonary or hepatitic arteriovenous fistula.

Angiogenesis is also involved in normal physiological processes, such as reproduction and wound healing. Angiogenesis is an important step in ovulation and also in implantation of the blastula after fertilization. Prevention of angiogenesis could be used to induce amenorrhea, to block ovulation, or to prevent implantation by the blastula. In wound healing, excessive repair or fibroplasia can be a detrimental side effect of surgical procedures and may be caused or exacerbated by angiogenesis. Adhesions are a frequent complication of surgery and lead to problems such as small bowel obstruction. Other angiogenesis dependent diseases of the reproductive system include endometriosis, ectopic pregnancy and uterine fibroids.

Diseases associated with chronic inflammation are accompanied by angiogenesis and can be treated by the compositions and methods of the present invention. Diseases with symptoms of chronic inflammation include obesity, inflammatory bowel diseases such as Crohn's disease and ulcerative colitis, psoriasis, sarcoidosis, atherosclerosis including plaque rupture, Sjogrens disease, acne rosacea, syphilis, chemical burns, bacterial ulcers, fungal ulcers, Behcet's syndrome, Stevens-Johnson's disease, Mycobacteria infections, Herpes simplex infections, Herpes zoster infections, protozoan infections, Mooren's ulcer, leprosy, Wegener's sarcoidosis, pemphigoid, lupus, systemic lupus erythematosis, polyarteritis, lyme's disease, Bartonelosis, tuberculosis, histoplasmosis and toxoplasmosis. Angiogenesis is a key element that these chronic inflammatory diseases have in common. The chronic inflammation depends on continuous formation of capillary sprouts to maintain an influx of inflammatory cells. The influx and presence of the inflammatory cells sometimes produce granulomas to help maintain the chronic inflammatory state. Inhibition of angiogenesis by the compositions and methods of the present invention would prevent the formation of the granulomas and alleviate the disease.

The inflammatory bowel diseases also show extraintestinal manifestations such as skin lesions. Such lesions are characterized by inflammation and angiogenesis and can occur at many sites other than the gastrointestinal tract. The compositions and methods of the present invention are also capable of treating these lesions by preventing the angiogenesis, thus, reducing the influx of inflammatory cells and the lesion formation.

Sarcoidosis is another chronic inflammatory disease that is characterized as a multisystem granulomatous disorder. The granulomas of this disease may form anywhere in the body, and, thus, the symptoms depend on the site of the granulomas and whether the disease active. The granulomas are created by the angiogenic capillary sprouts providing a constant supply of inflammatory cells.

Lastly, tissue and organ growth is regulated by the available vascular supply and new supply from angiogenesis. Thus obesity has been found to be inhibited by angiogenesis inhibitors since fat pad growth requires angiogenesis. Inhibitors of angiogenesis, as described herein, are contemplated for use in the treatment of obesity, for weight loss, for weight control, or for maintenance of a weight following e.g., surgery or dietary intervention (see e.g., U.S. Pat. No. 6,306,819, which is herein incorporated by reference in its entirety). Alternatively, activators of angiogenesis can be used to promote weight gain in e.g., anorexic or malnourished individuals.

Dosage and Administration

In one aspect, the methods described herein provide a method for treating an angiogenesis-associated disease in a subject. In one embodiment, the subject can be a mammal. In another embodiment, the mammal can be a human, although the approach is effective with respect to all mammals. The method comprises administering to the subject an effective amount of a pharmaceutical composition comprising an agent that inhibits fibromodulin activity, in a pharmaceutically acceptable carrier.

The dosage range for the agent depends upon the potency, and include amounts large enough to produce the desired effect, e.g., a reduction in invasion of new blood vessels in the eye or elsewhere. The dosage should not be so large as to cause unacceptable adverse side effects. Generally, the dosage will vary with the type of fibromodulin activity inhibitor (e.g., an antibody or fragment, small molecule, siRNA, etc.), and with the age, condition, and sex of the patient. The dosage can be determined by one of skill in the art and can also be adjusted by the individual physician in the event of any complication. Typically, the dosage ranges from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 μg/kg body weight. Alternatively, the dose range will be titrated to maintain serum levels between 5 μg/mL and 30 μg/mL.

As another alternative, dosage are selected for localized delivery and are not necessary selected to body weight or to achieve a certain serum level, but to achieve a localized effect, e.g., as for a localized injection, implantation or other localized administration to the eye.

Administration of the doses recited above can be repeated for a limited period of time. In some embodiments, the doses are given once a day, or multiple times a day, for example but not limited to three times a day. In a preferred embodiment, the doses recited above are administered daily for several weeks or months. The duration of treatment depends upon the subject's clinical progress and responsiveness to therapy. Continuous, relatively low maintenance doses are contemplated after an initial higher therapeutic dose.

A therapeutically effective amount is an amount of an agent that is sufficient to produce a statistically significant, measurable change in neovascular formation, number of blood vessels etc. (see “Efficacy Measurement” below). Such effective amounts can be gauged in clinical trials as well as animal studies for a given fibromodulin activity inhibitor.

Agents useful in the methods and compositions described herein can be administered topically, intravenously (by bolus or continuous infusion), orally, by inhalation, intraperitoneally, intramuscularly, subcutaneously, intracavity, and can be delivered by peristaltic means, if desired, or by other means known by those skilled in the art. It is preferred that the agents for the methods described herein are administered topically to the eye. For the treatment of tumors, the agent can be administered systemically, or alternatively, can be administered directly to the tumor e.g., by intratumor injection or by injection into the tumor's primary blood supply.

Therapeutic compositions containing at least one agent can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology. In some embodiments, a fibromodulin activity inhibitor can be targeted to tissue- or tumor-specific targets by using bispecific antibodies, for example produced by chemical linkage of an anti-ligand antibody (Ab) and an Ab directed toward a specific target. To avoid the limitations of chemical conjugates, molecular conjugates of antibodies can be used for production of recombinant bispecific single-chain Abs directing ligands and/or chimeric inhibitors at cell surface molecules. The addition of an antibody to a fibromodulin activity inhibitor permits the agent attached to accumulate additively at the desired target site. Antibody-based or non-antibody-based targeting moieties can be employed to deliver a ligand or the inhibitor to a target site. Preferably, a natural binding agent for an unregulated or disease associated antigen is used for this purpose.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood in the ranges specified for in vivo therapies are contemplated.

An agent may be adapted for catheter-based delivery systems including coated balloons, slow-release drug-eluting stents or other drug-eluting formats, microencapsulated PEG liposomes, or nanobeads for delivery using direct mechanical intervention with or without adjunctive techniques such as ultrasound.

In some embodiments, an inhibitor may be combined with one or more agents such as chemotherapeutic or anti-angiogenic agents, for the treatment of an angiogenesis associated disease.

Pharmaceutical Compositions

The present invention involves therapeutic compositions useful for practicing the therapeutic methods described herein. Therapeutic compositions contain a physiologically tolerable carrier together with an active agent as described herein, dissolved or dispersed therein as an active ingredient. In a preferred embodiment, the therapeutic composition is not immunogenic when administered to a mammal or human patient for therapeutic purposes. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Specifically contemplated pharmaceutical compositions are active RNAi ingredients in a preparation for delivery as described herein above, or in references cited and incorporated herein in that section. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques.

Efficacy measurement

The efficacy of a given treatment for an angiogenesis-associated disease can be determined by the skilled clinician. However, a treatment is considered “effective treatment,” as the term is used herein, if any one or all of the signs or symptoms of, as but one example, ocular neovascular disease are altered in a beneficial manner, other clinically accepted symptoms or markers of disease are improved, or even ameliorated, e.g., by at least 10% following treatment with a fibromodulin inhibitor. Efficacy can also be measured by a failure of an individual to worsen as assessed by hospitalization or need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the pathogenic growth of new blood vessels; or (2) relieving the disease, e.g., causing regression of symptoms, reducing the number of new blood vessels in a tissue exhibiting pathology involving angiogenesis (eg., the eye); and (3) preventing or reducing the likelihood of the development of a neovascular disease, e.g., an ocular neovascular disease).

An effective amount for the treatment of a disease means that amount which, when administered to a mammal in need thereof, is sufficient to result in effective treatment as that term is defined herein, for that disease. Efficacy of an agent can be determined by assessing physical indicators of, for example ocular neovascular disease, such as e.g., visual problems, new blood vessel invasion, rate of vessel growth, angiogenesis, etc.

For treatment of a subject with a fibromodulin polypeptide (or other fibromodulin activity activator), the in vivo efficacy of an agent can be measured by e.g., rate of wound healing, size of scar, increased fertility, as well as by assessing an increase in various markers of angiogenesis as described herein.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

The present invention may be as defined in any one of the following numbered paragraphs.
1. A fibromodulin activity inhibitor for use in the treatment of an angiogenesis-related disease.
2. The use of paragraph 1, wherein the fibromodulin activity inhibitor is selected from the group consisting of an antibody, an RNA interference molecule, a small molecule, a peptide and an aptamer.
3. The use of paragraph 2, wherein the fibromodulin activity inhibitor comprises an antibody.
4. The use of paragraph 1, wherein the angiogenesis-related disease is age-related macular degeneration.
5. Use of a fibromodulin activity inhibitor in the manufacture of a medicament for the treatment of an angiogenesis-related disease.
6. The use of paragraph 5, wherein the fibromodulin activity inhibitor is selected from the group consisting of an antibody, an RNA interference molecule, a small molecule, a peptide and an aptamer.
7. The use of paragraph 6, wherein the fibromodulin activity inhibitor comprises an antibody.
8. The use of paragraph 5, wherein the angiogenesis-related disease is age-related macular degeneration.
9. Use of a fibromodulin polypeptide or fragment thereof in the manufacture of a medicament for the treatment of a disorder of impaired angiogenesis.
10. The use of paragraph 9, wherein the disorder is impaired response to wound healing.
11. The use of paragraph 9, wherein the disorder is impaired fertility.
12. Use of a fibromodulin polypeptide or fragment thereof in the manufacture of a medicament for the treatment of a wound.
13. A method for inhibiting endothelial cell proliferation and/or migration, the method comprising contacting an endothelial cell with a fibromodulin activity inhibitor.
14. The method of paragraph 13, wherein the fibromodulin activity inhibitor is selected from the group consisting of an antibody, an RNA interference molecule, a small molecule, a peptide and an aptamer.
15. The method of paragraph 13, wherein the fibromodulin activity inhibitor comprises an antibody.
16. The method of paragraph 13, wherein the cell is selected from the group consisting of a primary cell or a cell of a cell line.
17. The method of paragraph 13, wherein the cell is human.
18. A method of treating an angiogenesis-related disease, the method comprising administering a therapeutically effective amount of a fibromodulin activity inhibitor to a mammal having an angiogenesis-related disease.
19. The method of paragraph 18, wherein the angiogenesis-related disease is age-related macular degeneration.
20. The method of paragraph 18, wherein the fibromodulin activity inhibitor is selected from the group consisting of an antibody, an RNA interference molecule, a small molecule, a peptide and an aptamer.
21. The method of paragraph 18, wherein the fibromodulin activity inhibitor comprises an antibody.
22. The method of paragraph 18, wherein the mammal is a human.
23. A method for inhibiting angiogenesis, the method comprising administering a therapeutically effective amount of a fibromodulin activity inhibitor to a mammal in need thereof.
24. The method of paragraph 23, wherein the fibromodulin activity inhibitor is selected from the group consisting of an antibody, an RNA interference molecule, a small molecule, a peptide and an aptamer.
25. The method of paragraph 23, wherein the fibromodulin activity inhibitor comprises an antibody.
26. The method of paragraph 23, wherein the mammal is a human.
27. A method for inhibiting fibromodulin activity in a mammal, the method comprising administering a therapeutically effective amount of a fibromodulin activity inhibitor to a mammal in need thereof.
28. The method of paragraph 27, wherein the fibromodulin activity inhibitor is selected from the group consisting of an antibody, an RNA interference molecule, a small molecule, a peptide and an aptamer.
29. The method of paragraph 28, wherein the fibromodulin activity inhibitor comprises an antibody.
30. The method of paragraph 27, wherein the mammal is a human.
31. A method for promoting endothelial cell proliferation and/or migration, the method comprising: contacting an endothelial cell with an agent that activates fibromodulin activity.
32. The method of paragraph 31, wherein the agent is a fibromodulin polypeptide, or a fragment thereof.
33. The method of paragraph 31, wherein the cell is selected from the group consisting of a primary cell or a cell of a cell line.
34. The method of paragraph 31, wherein the cell is human.
35. A method for promoting angiogenesis in a subject, the method comprising: administering a therapeutically effective amount of an agent that activates fibromodulin activity to a subject in need thereof.
36. The method of paragraph 35, wherein the agent is a fibromodulin polypeptide, or a fragment thereof.
37. The method of paragraph 35, wherein the subject is a mammal.
38. The method of paragraph 37, wherein the mammal is a human.
39. A method for promoting wound healing in a subject, the method comprising: administering an agent that activates fibromodulin activity.
40. The method of paragraph 39, wherein the agent is a fibromodulin polypeptide, or a fragment thereof.
41. The method of paragraph 39, wherein the subject is a mammal.
42. The method of paragraph 41, wherein the mammal is a human.

### EXAMPLES

Fibromodulin is a member of a family of small interstitial proteoglycans with leucine-rich repeats. The inventors have discovered a novel property of fibromodulin that relates to the proliferation and migration of vascular endothelial cells. Promoting vascular growth is useful for the treatment of disorders with insufficient angiogenesis such as wound healing. Further the inventors have found that inhibitors of fibromodulin, including antibodies to fibromodulin can significantly inhibit endothelial cell proliferation and can thus serve as treatments to inhibit angiogenesis dependent diseases such as macular degeneration and cancer, among others.

The term “pathological angiogenesis” refers to the excessive formation and growth of blood vessels during the maintenance and the progression of several disease states. Examples where pathological angiogenesis can occur are found in ocular disorders, such as age related macular degeneration and diabetic retinopathy, as well as in many other disorders such as cancer and arthritis.

The role of FMOD in vasculogenesis in the developing embryo and postnatal angiogenesis has not yet been identified in literature. Through detailed in vitro and in vivo studies of fibromodulin, the inventors have found that FMOD plays a key role in pathological angiogenesis and that inhibitors of FMOD can be used to prevent blood vessel formation.

### Example 1

### Pro-Angiogenic Effects of Fibromodulin

FMOD affects migration of endothelial cells (EC).

Cell migration is a fundamental function of normal cellular processes, including embryonic development, angiogenesis and wound healing. A standard migration assay was used to measure the migration of cells through a membrane. The cells which migrate through the membrane are dissociated from the membrane and counted using the CyQuant GR dye (molecular probe). This fluorescent dye binds to nucleic acids and gives an increasing signal with increasing cell number.

Eight replicate samples of endothelial cells (100,000/ml) per condition were seeded into the upper chamber of transwells in the presence of full conditioned medium (CM) from melanocytes as a positive control. Negative control received starving conditioned medium, 40 nM recombinant FMOD protein or GST was added to the starving condition in order to determine their effect on migration. After 4 hours, cells on the lower surface were detached, lysed, incubated with the dye and read with a fluorescence reader using 480/520 nm filter. Significant differences in migration are indicated where P<0.001 using Student's t-test.

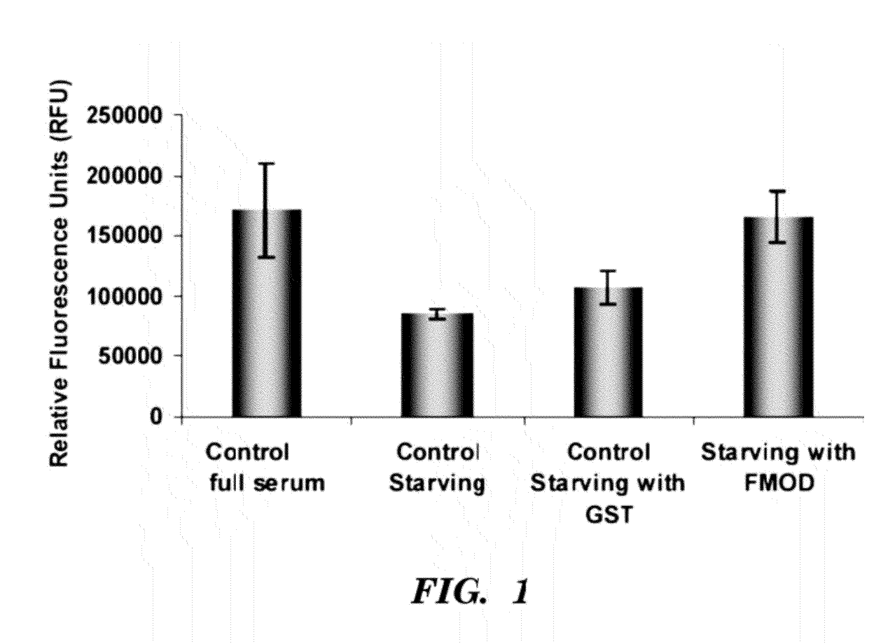

As indicated in FIG. 1, the CM contained an agent that significantly increased the migration of ECs in the transwell experiment. The migration promoting agent is absent in the CM of starved cells. Addition of 40 nM recombinant FMOD protein to the CM of starved cells produces the same migration induction effect as observed for the non-starved full CM.

FMOD affects endothelial cell proliferation.

Cell proliferation was assessed by staining proliferating cells in an assay based on the cleavage of the tetrazolium salt WST-1 to formazan by cellular mitochondrial dehydrogenases. 5000 cells were plated in each well of a 96 well plate overnight. All wells were rinsed with phosphate-buffered saline. Negative control wells received starvation medium, and positive control wells received full medium. 40 nM recombinant FMOD protein or GST as a control was added to the starving condition in order to determine their effect on proliferation. Cells were allowed to incubate for 24 h. At this time WST-1 reagent was applied for 4 h to measure cell proliferation. The cells were read using a 450 nm filter. Significant differences in proliferation are indicated where P<0.001 using Student's t-test.

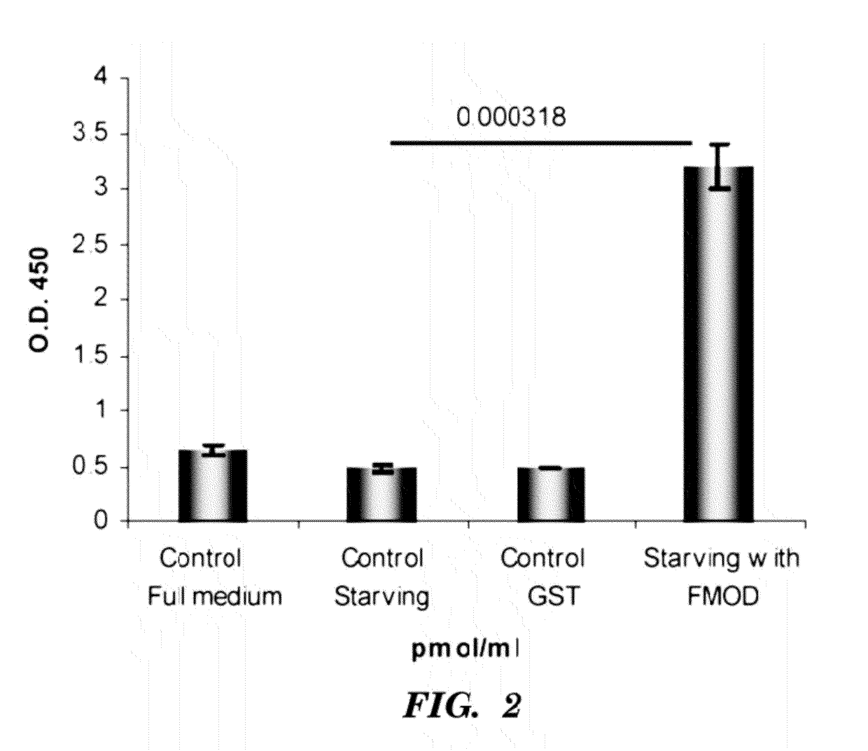

Expansion in the number of viable cells results in an increase in the overall activity of the mitochondrial dehydrogenases in the sample. As shown in FIG. 2, human microvessel endothelial cells, dermal (HMVEC-d) cell proliferation is significantly increased by incubation with recombinant FMOD (p<0.001).

Fibromodulin increases endothelial cell migration in vivo as tested in the Matrigel assay.

Angiogenesis was studied in vivo in a matrigel assay performed as described. Two groups of 8-week-old C57BL/6 mice were injected subcutaneously with matrigel containing 6.5 pmol FMOD, or 6.5 pmol GST as control. On day 6, animals were sacrificed and fluorescence-activated cell sorting (FACS) analysis was used for determination of the matrigel liberated cells. In order to distinguish the endothelial from hematopoietic cells the inventors used the antibodies CD31-PE, and CD45-APC respectively.

In vitro 3-D Sprouting assay

HMVECs were cultured on Cytodex-3; microcarrier beads (Amersham Biosciences) coated with fibrinogen at a ratio of 200 cells per bead. Cells and beads were suspended in 5 ml basal medium (EBM) mixed gently every hour for the first 4 h; the mixture was then cultured overnight at 37° C. The next day, HMVEC coated bead culture was embedded in 1 mg/ml fibrin gel with EBM medium with/without recombinant FMOD (1.5 nM or 4.6 nM) in 48-well plate for two days. A combination (combo) of angiogenic factors FGF2 (1.5 nM) and vascular endothelial growth factor-A (VEGFA, 1.5 nM) was used as the positive angiogenic control The effect of the pro-angiogenesis factors on sprout angiogenesis was quantified visually by determining the number and percent of HMVEC-beads with capillary sprouts.

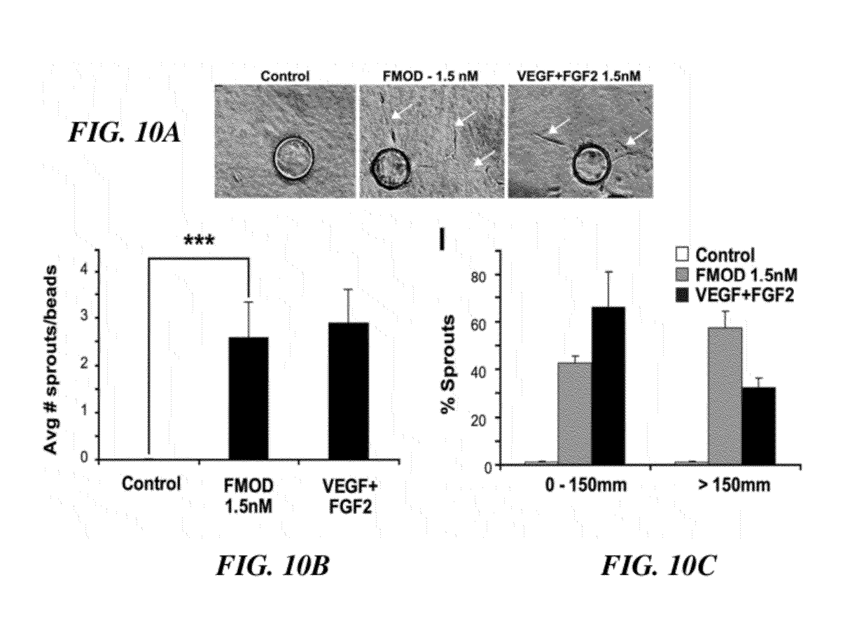

As seen in FIG. 10A, sprouts (arrow) are observed in HMVEC coated beads that are embedded into a fibrin gel in the presence of medium alone or 1.5 nM FMOD, or a mixture of VEGF/FGF2 (1.5 nM each).

FIG. 10B indicates that both FMOD and the “combo” (VEGF+FGF) were comparable in inducing sprouting of HMVEC structures from the HMVEC coated beads. The average numbers of sprouts per bead was not significantly different between 1.5 nM FMOD (75 sprouts) and positive VEGFA/FGF2 control (68 sprouts). Significant differences were observed between recombinant FMOD and control; p<0.0001.

However, FMOD induced longer sprouts compared to the “combo”. The percentage of sprouts with a length up to 150 μm in the presence of 1.5 nM FMOD (43%), and 1.5 nM VEGFA+FGF2 (68%) were also determined. Sprout length was greater with 150 μm FMOD, revealing 57% versus 32% for 1.5 nM VEGFA/FGF2. 1.5 nM FMOD induced 1.9 and 1.6 times further cell migration than VEGFA/FGF2, respectively; p<0.001.

Model of Dermatological Wound Healing.

Vascular remodeling was studied in a model of skin wound healing, since it is dependent on neovascularization. The size of the wounds was standardized through the use of a biopsy punch which creates a circular wound measuring 1 mm. A full-thickness skin incision was made on the dorsal aspect of the mouse ear. Wound healing was observed and imaging analyzed throughout the entire healing process by daily measuring the width and the length of the wound. New blood vessel formation was analyzed in vivo and on skin sections harvested five days after wounding.

In order to evaluate wound neovascularization, mouse ears were subjected to full thickness skin punch and then treated with GST as control or FMOD (P is for the Punch). To visualize the blood vessels mice were injected with dextran-FITC. Vessels were measured by pixels using by ImageJ.

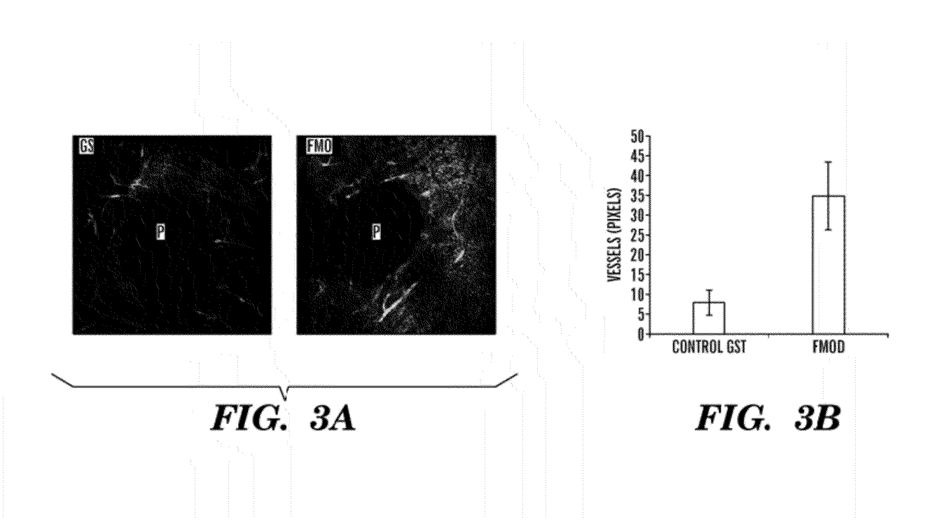

The ears of mice were treated daily with either matrigel, or matrigel containing GST or FMOD (8 nM) respectively, over the course of five days. In order to evaluate the wound neovascularization, mice were anesthetized and injected with dextran-FITC to label specifically the endothelial cells. Results are shown in FIG. 3; the angiogenesis around the circular wound among the treated mice was visualized with intravenous dextran-FITC.

The ear/wound was observed and is shown in FIG. 3A. The angiogenesis around the circular wound among mice treated with recombinant FMOD was higher than among mice treated with recombinant FMOD (p<0.001). Quantitation of these observations is presented in FIG. 3B.

Fibromodulin injected into the vitreous dramatically increases in vivo angiogenesis in a mouse model of Choroidal neovascularization (CNV).

To evaluate how Fibromodulin modulates the angiogenic process in the eye, a choroidal neovascularization assay was performed. CNV is an abnormal vessel growth from the choriocapillaris through Bruch's membrane resulting in hemorrhage, scarring, exudation, and/or retinal detachment, with the ultimate consequence of a severe loss of high-acuity central vision. It is the leading cause of blindness seen in age-related macular degeneration.

Experiments were performed to investigate the effect of intravitreous injections of GST (5.25 nM) and FMOD (5.25 nM) on laser-induced CNV. Briefly, for the assay, 6-8 week-old C57BL/6J mice were anesthetized followed by 1% Tropicamide for pupillary dilation. Three burns of 532-nm diode laser photocoagulation (50-μm spot size, 0.1-s duration, 200 mW) were performed. Animals were injected immediately after laser injury into the vitreous with the fibromodulin or GST polypeptide (n=15-18 successful burns in each group). One week later, mice were anesthetized and perfused with fluorescein-labeled dextran (1×10⁶ average molecular weight) (SIGMA-ALDRICH®, St. Louis, Mo.). Eyes were enucleated and labeled with anti-Lectin and or anti-CD31PE (INVITROGEN™). Choroidal flat mounts were prepared, and the CNV area was measured as described previously.

Five mice were used for each group with three burns in each eye (n=15-18 successful burns in each group). For quantitative analysis of lesion intensity and size, CNV images were batch-processed by using imageJ. Perimeter, area, and mean diameter (pixels) were calculated for each region of interest and exported to Excel (MICROSOFT, Redmond, Wash.). Analyses were performed by a blind observer to eliminate user bias.

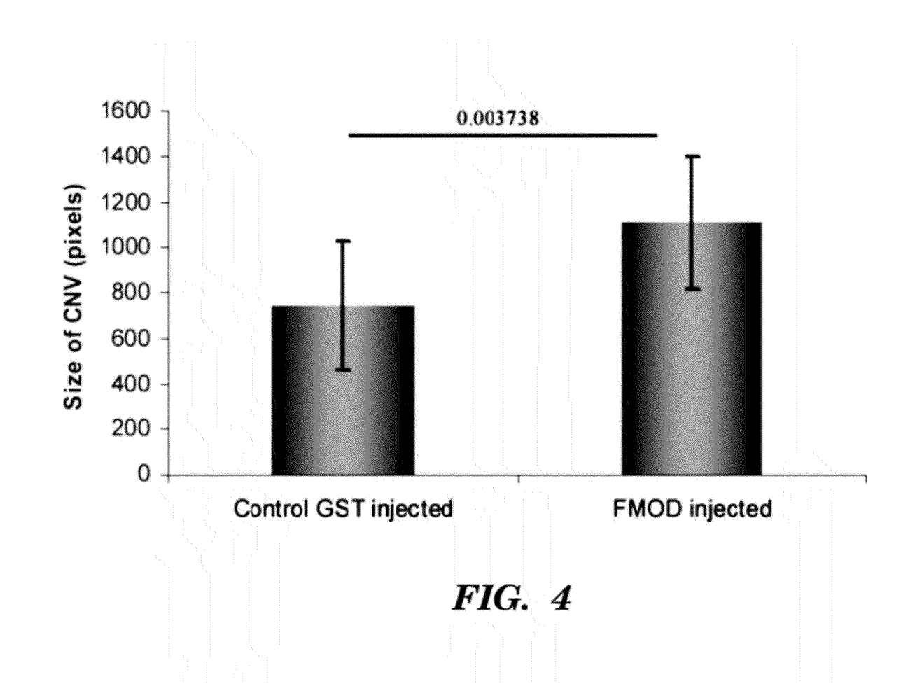

These data indicate that a significant effect of fibromodulin on CNV was observed (FIG. 4).

Additional laser-induced CNV experiments were conducted to assess the effects of FMOD in CNV progression. The laser-induced CNV was generated by a previously described technique with some modifications. C57B1/6J mice were anesthetized with avertin (400 mg/kg). A mixture of tropicamide (each 0.5%), and phenylephrine hydrochloride (Mydrin P; Santen Pharmaceutical, Osaka, Japan) was applied to both eyes to dilate the pupils. Lesions were induced by a diode pumped solid state laser (0.1 s; spot size, 50 μm; power 150 mW) around the optic nerve through a slit lamp delivery system using a Nidek photocoagulator (GYC2000, Nidek, Osaka, Japan). Only lesions in which a subretinal bubble or focal serous detachment of the retina developed were used for the experiments. FMOD (100 ng/0.5 ul) was administered by intravitreous injection on the day of CNV induction (day 0) and the sizes of CNV lesions were determined after 14 days. After 14 days post-laser induction, mice were euthanized and their eyes were removed and fixed in 4% paraformaldehyde for 60 min. The retina was flat mounted and the blood vessels were labeled using lectin-FITC (Vector Laboratories). Fluorescent images of choroidal flat-mounts were captured using a CCD camera (DC500, Leica, Switzerland). The CNV area was evaluated using Scion image software.

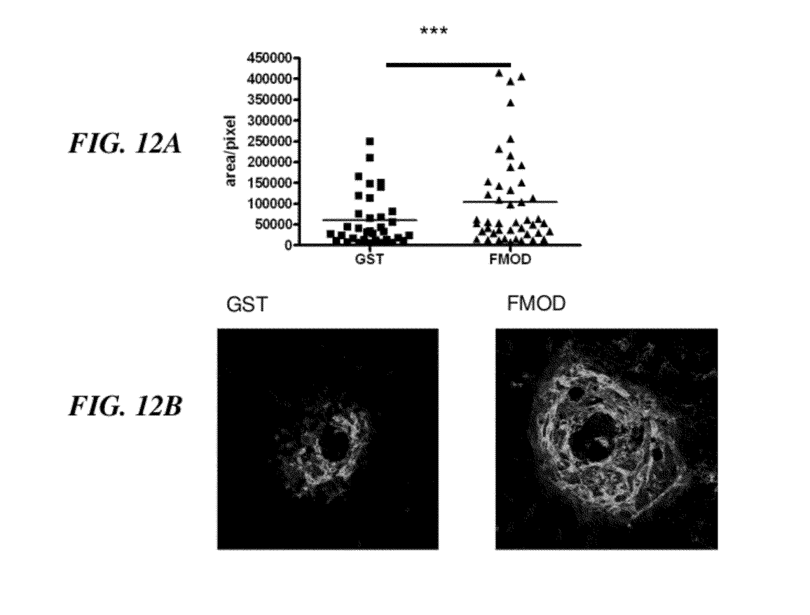

In the data shown in FIG. 12, CNV lesions were induced by laser around the optic nerve through a slit lamp delivery system. Only lesions in which a subretinal bubble or focal serous detachment of the retina developed were used for the experiments. Four burns were performed per eye while leaving a space around the optic disc. At the day of laser induction intravitreal injection of 100 ng/0.5 ul recombinant FMOD or control-GST was performed. FIG. 12A presents the data as mean pixel number±SEM (n=30-40, *P<0.0005, U-test). There is an increase of over 42% in in vivo angiogenesis in the FMOD treated mouse compared to the control GST treated mouse.

FIG. 12B provide representative images of retinal flat-mounts stained with a lectin-FITC, Bars=10 μm, P<0.0005.

### Example 2

### siRNA-FMOD Reduces Migration of Endothelial Cells

A standard migration assay, which measures the migration of cells through a membrane, was used to determine the effect of fibromodulin inhibition in cells. The cells which migrate through the membrane are dissociated from the membrane and counted using the CyQunant GR dye (MOLECULAR PROBE, INVITROGEN™). This fluorescent dye binds to nucleic acids and gives an increasing signal with increasing cell number. A period of 48 hr before the migration assay, non-pigmented melanocytes were transfected with pre-designed siRNA (AMBION®) against FMOD transcript or scrambled siRNA without significant homology to human gene sequences as a control. Eight replicate samples of cells (100,000/mL) per condition were seeded into the upper chamber of transwells in the presence of conditioned medium from the transfected cells. Dye was added to the cells in the upper chamber and the dyed cells were read with a fluorescence reader using 480/520 nm filter. Significant differences in migration are indicated where P<0.001 using Student's t-test.

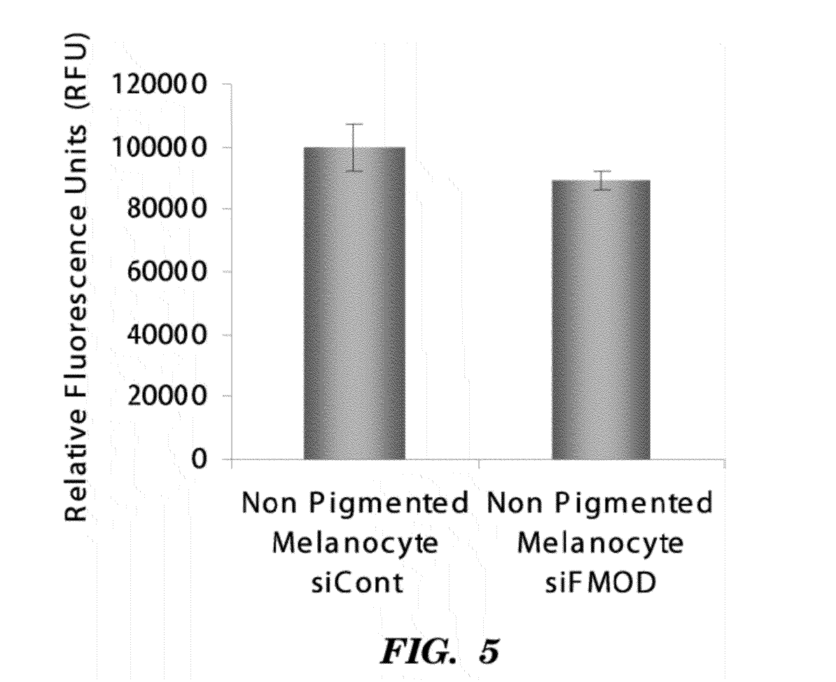

FIG. 5 shows that siRNA-fibromodulin reduces migration of human microvessel endothelial cells.

Anti-Fibromodulin decreases endothelial cell migration in vivo as tested in a MATRIGEL™ assay

Angiogenesis was studied in vivo in a MATRIGEL™ assay performed as described.

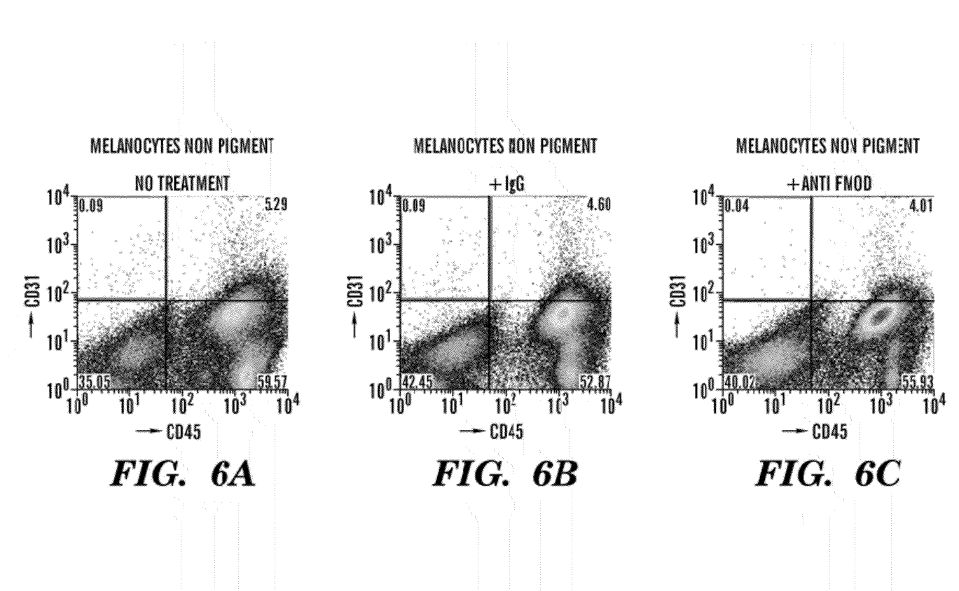

Three groups of 8-week-old C57BL/6 mice were injected subcutaneously with non-pigmented melanocytes mixed with MATRIGEL™ containing anti FMOD antibody, or IgG-goat or cells alone. On day 6, animals were sacrificed and the implanted MATRIGEL™ was extracted and digested to liberate the cells that have migrated into the gel matrix while in vivo. Fluorescence-activated cell sorting (FACS) analysis was used to determine the number of MATRIGEL™ liberated cells. In order to distinguish the endothelial from hematopoietic cells the following antibodies were used: CD31-PE (endothelial cell marker), and CD45-APC (hematopoietic cell marker). As shown in FIG. 6, 2.25 times more endothelial cells (0.09% versus 0.04%) (upper left panel) infiltrated the MATRIGEL™ plugs compared to when only cells or cells with IgG were included. This observation confirms the hypothesis that FMOD is a potent angiogenic factor which has a synergistic effect on endothelial cell migration.

### Example 3

### Corneal Micropocket Assay

To further evaluate the role of fibromodulin as an angiogenic regulator, a corneal micropocket assay was performed. The corneal micropocket assay was performed as described, using pellets containing 4 μM of FMOD, 4 μM GST or 4 μM carrier-free human recombinant VEGF 165. The area of vascular response was assessed on the sixth post-operative day using a slit lamp. Vessel area in the cornea was calculated using the equation 0.2×VL×CH, where VL is vessel length from the limbus in millimeters and CH is clock hours around the cornea. In order to inhibit the angiogenesis, mice were injected intraperitoneally everyday with 1 mg/kg antibody against FMOD (anti-FMOD) or IgG as control.

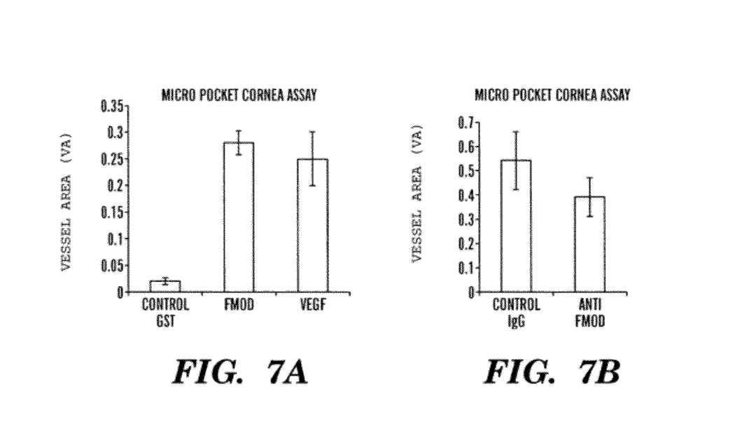

Results are shown in FIG. 7. FIG. 7A indicates that fibromodulin promotes angiogenesis in the cornea and the pro-angiogenic activity of fibromodulin is comparable to iVEGF. FIG. 7B shows that anti-FMOD inhibited angiogenesis in the cornea.

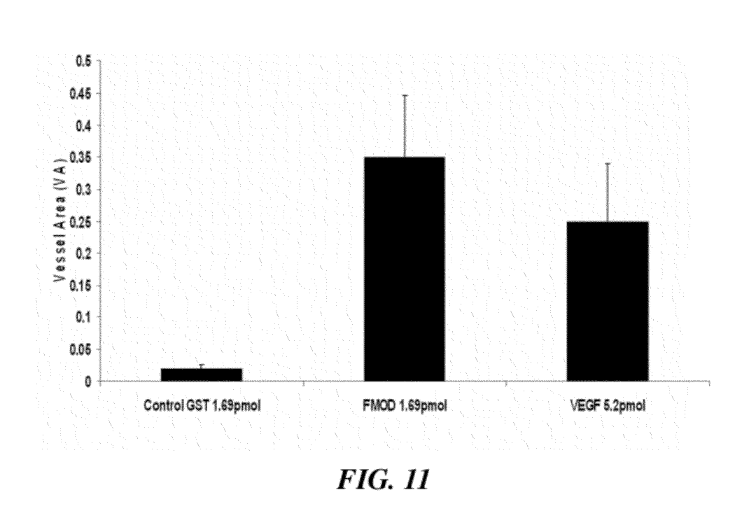

Similarly in FIG. 11, the corneal micropocket assay was performed with pellets containing 1.69 pmol of FMOD, 1.69 pmol GST, or 5.2 pmol VEGFA. The comparison of FMOD group to the control group indicates that the FMOD group was significantly different p<0.0001. No significant difference was found in vessel area between the FMOD group and the VEGF group.

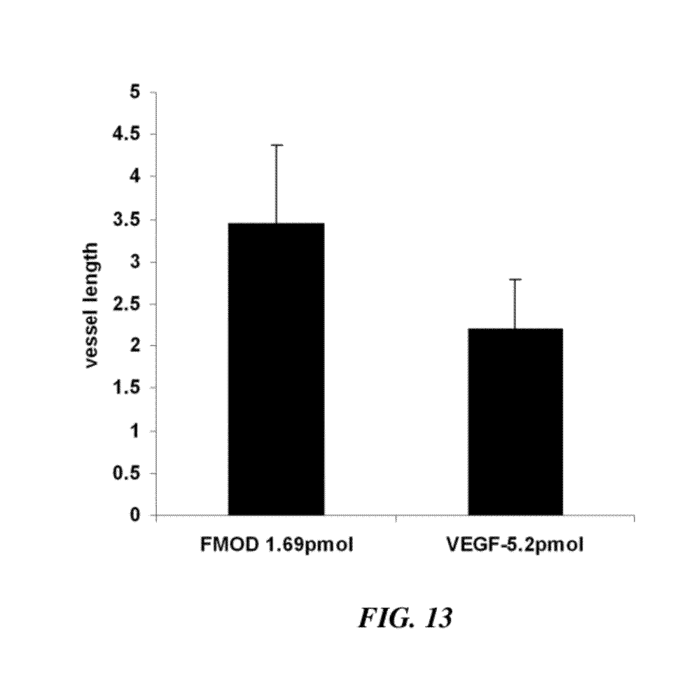

When the amount of induced angiogenesis is quantified according to the vessel length, FMOD induces longer vessel length than VEGF (FIG. 13).

Retinal neovascularization in young pups

Quantification of retinal neovascularization was performed 7 days after birth. Mosaic images covering the entire retina at 5× magnification were taken on a fluorescence microscope. 4 day old pups were injected intravitreally with recombinant FMOD (0.5 μM) versus GST (control); fluorescence micrographs (Axio Observer Z1Zeiss) on day 7 show projected images of vessels stained with Alexa-594-isolectin in the retina. Neovascular tuft formation was quantified by comparing the number of pixels in the affected areas with the total number of pixels in the retina (Image J). Digitized images of the total retinal area and peripheral avascular areas were measured using the freeware ImageJ. The peripheral avascular area was expressed as a percentage of the total retinal area. Neovascularization density was quantified by summing capillary junctions within four equal areas, in each of the four quadrants of the vascularized retina determined by ImageJ. Areas of VO and NV were quantified as percentages of total retina area. P7, n=10; P<0.001.

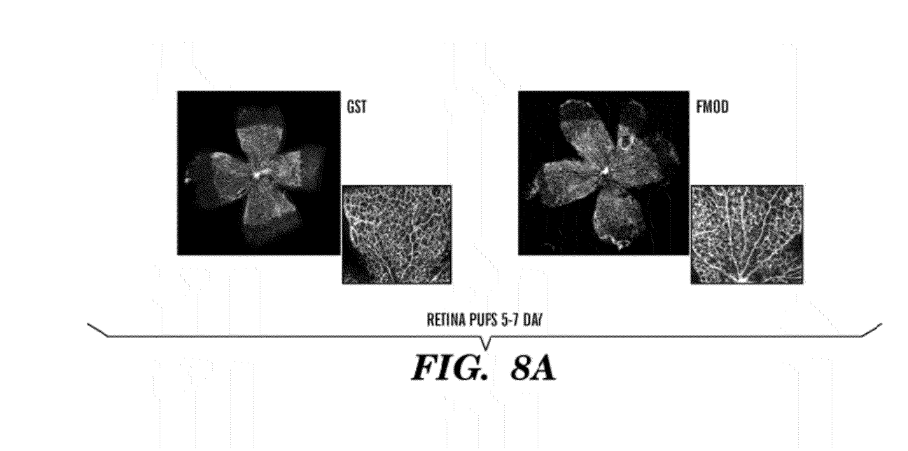

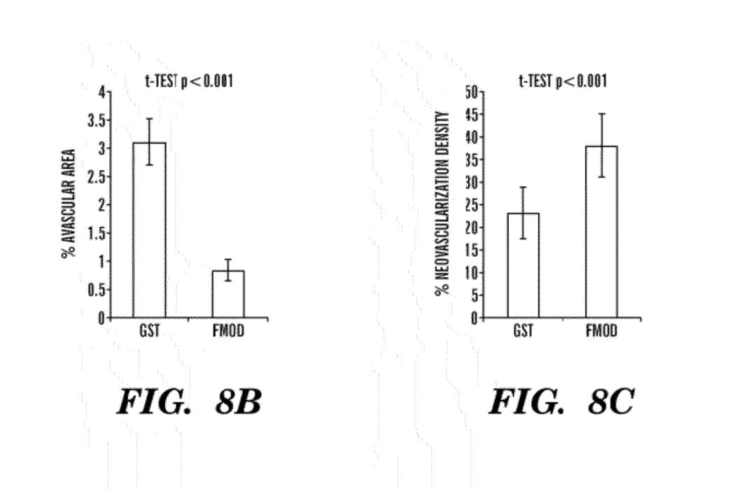

The peripheral avascular area was expressed as a percentage of the total retinal area. Results are shown in FIG. 8, indicating that pups treated with fibromodulin have a lower percent avascular area and an increased percent neovascularization compared to pups not treated with fibromodulin. These results indicate that fibromodulin promotes angiogenesis and formation of new blood vessels in the retina.

### Example 4

### Effects of Fibromodulin on Tumor Growth

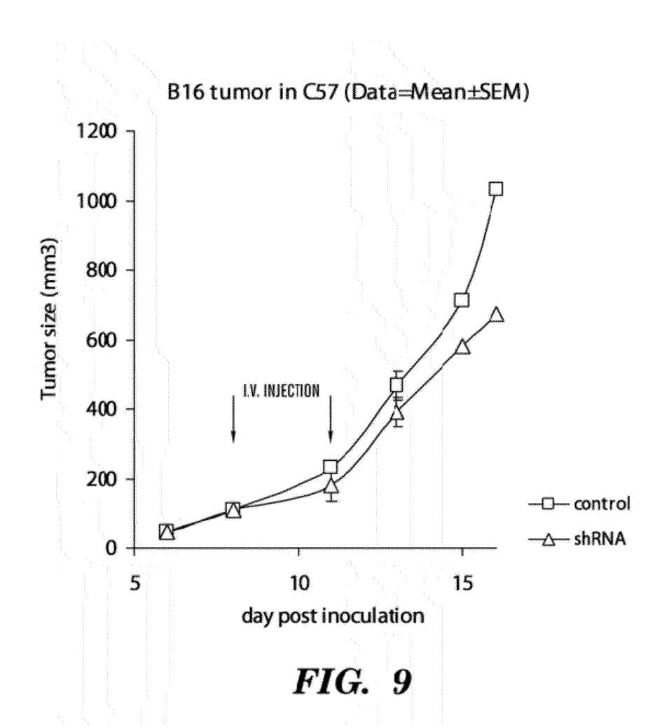

To confirm that fibromodulin plays a role in the microenvironment during tumor development, fibromodulin expression was systemically inhibited by inoculation of mice with fibromodulin siRNA or a control siRNA. In vivo experiments were obtained by intradermal injection of 1×10⁶ melanoma cells (B16Luc) on the rear dorsum of 6 week-old male C57BL/6J mice. Mice received 2 tail vein injections of either 5 nmol siRNA-FMOD or 5 nmol siRNA scramble on day 8 and day 11. The siRNA was complexed with a polymer from TRANSIT™ in vivo gene delivery system according to the manufacturer's recommendations (TRANSIT-QR™ Hydrodynamic Delivery System—Mirus). Mice were treated once the tumors reached a volume of 100-150 mm³. FIG. 9 shows the results of treatment with siRNA FMOD on the tumor growth.

### Example 5

### Fibromodulin (FMOD) Expression by Melanocytes

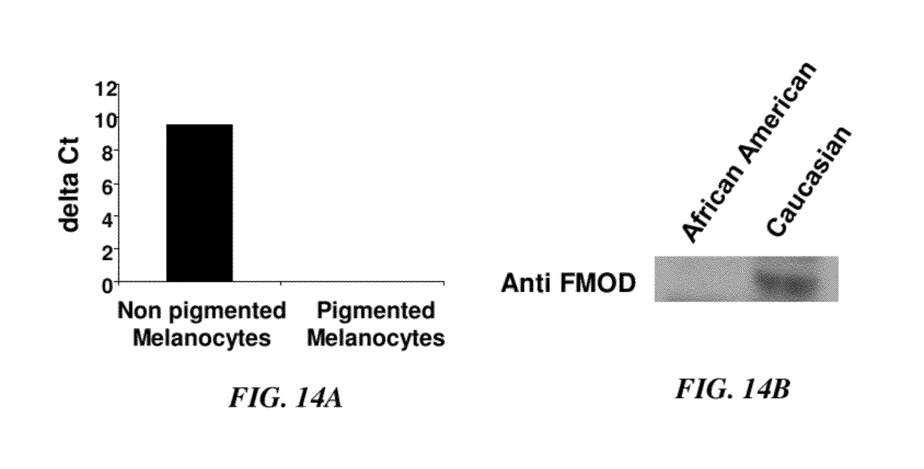

Comparison of the relative expression of FMOD mRNA and protein in cultured pigmented and non-pigmented mouse melanocytes was performed by real-time RT-PCR analysis (FIG. 14A), by in situ immunofluorescent staining (data not shown) and by Western Blot (FIG. 14B). The albino, non-pigmented mouse melanocytes express dramatically more (>100 fold) FMOD mRNA than pigmented melanocytes (see FIG. 14A and 14B).

Localization of FMOD expression in the retina

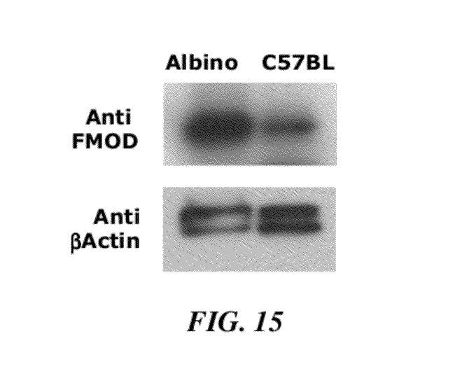

In order to determine the location of the expression of FMOD in vivo, in situ immunofluorescent staining of the retina was performed. Staining for FMOD in cryosections from C57-Albino and C57B1 mouse retinas were performed and compared. Strong FMOD staining was seen in the choroid (the region of the retina with melanocytes) (data not shown). In addition, FMOD levels were also assessed by Western Blot analysis in isolated choroids. FIG. 15 indicates that the choroid of albino mice expressed significantly more FMOD than the choroid of normal mice.

Stimulation of endothelial cell (EC) migration by conditioned media (CM) from melanocytes due to the expression of FMOD

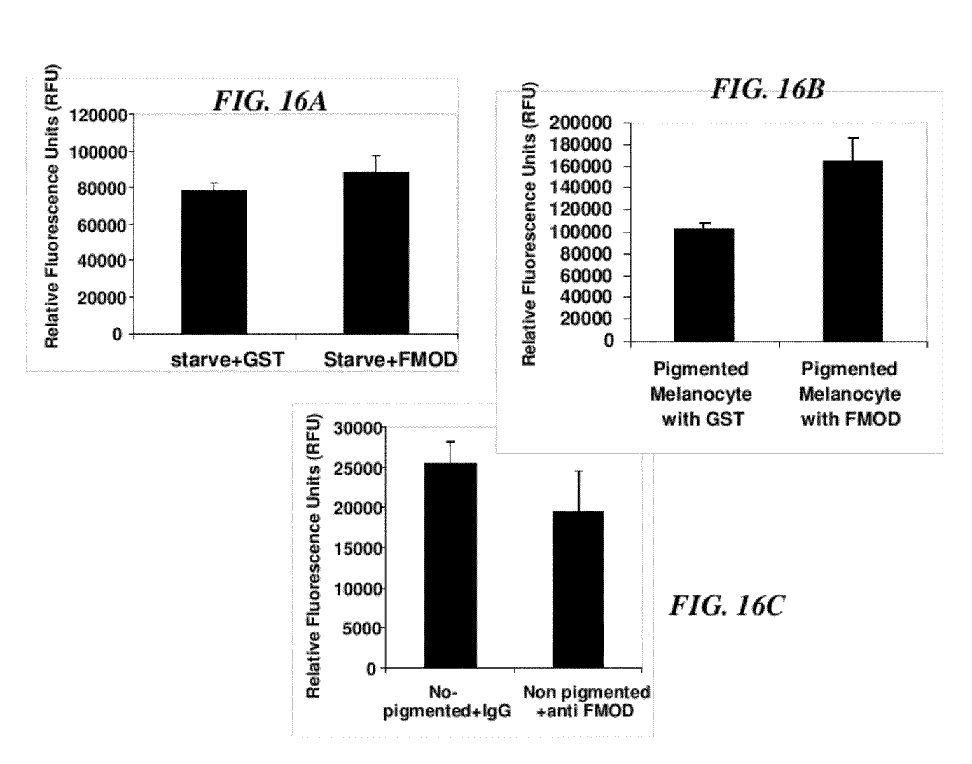

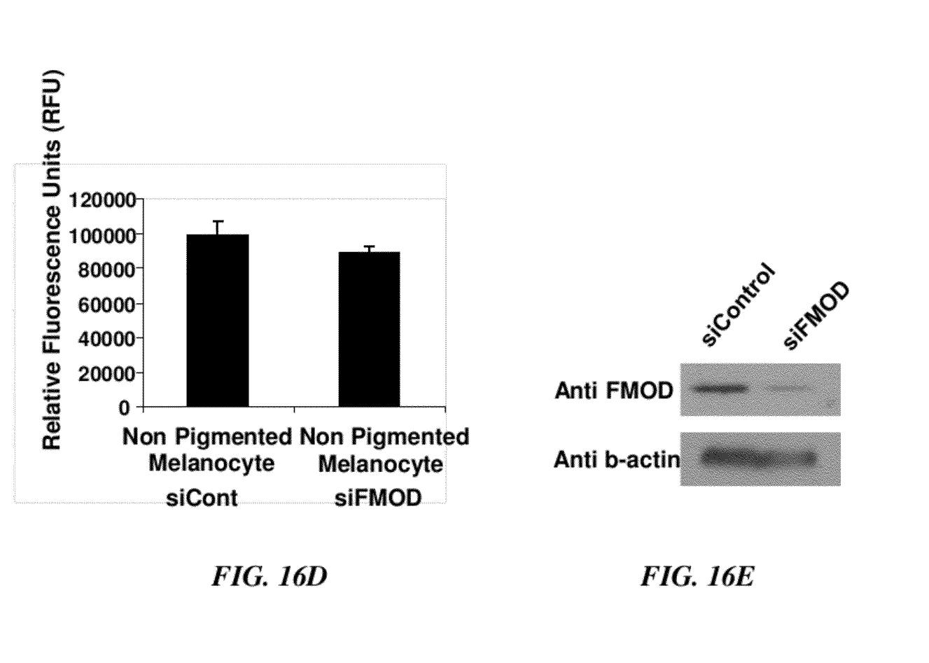

FIG. 16A shows the migration of EC induced by CM from starved pigmented melanocytes wherein the CM has been supplemented with GST as control or with recombinant FMOD (rFMOD or rhFMOD). The presence of FMOD increased the amount of EC migration compared to the control GST.

FIG. 16B shows the migration of EC induced by CM from pigmented melanocytes wherein the CM has been supplemented with GST or with rFMOD. The addition of rFMOD increased the amount of EC migration compared to the control GST.

FIG. 16C shows the migration of EC induced by CM from non-pigmented melanocytes wherein the FMOD has been neutralized. The CM was pre-treated with anti-FMOD antibody before use in the induction migration experiment to remove the FMOD.

FIG. 16D shows the migration of EC is reduced when FMOD expression is knocked down by siRNA in the non-pigmented melanocytes.

FIG. 16E shows that the expression of FMOD in the non-pigmented melanocytes, as confirmed by Western Blot, was effectively silenced by siRNA to FMOD

Effects of FMOD knockdown demonstrated in an in vivo MATRIGEL™ angiogenesis assay

B6 (Cg)-Tyrc-2J/J (C57-Albino) (8 weeks old) (Jackson Laboratories, Bar Harbor, Me.), or Tyrc-2J/J/FMOD KO (KO-FMOD Albino mice) mice were inoculated subcutaneously with MATRIGEL™ Matrix (BD Bioscience) mixed with recombinant human FGF-2 500ng/ml (R&D Systems). After 7 days, Matrigel plugs were removed, Immunostaining was performed to quantify endothelial cell migration.

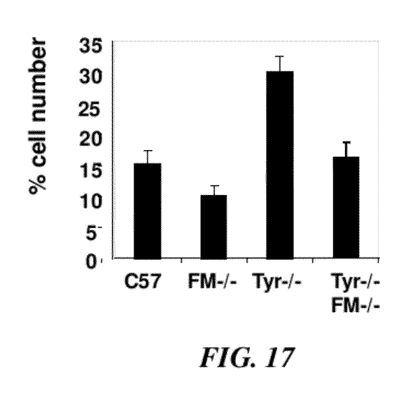

FIG. 17 shows the migration of host EC into MATRIGELTM plugs containing 500 ng FGF-2 for the four types of mice tested: C57 (normal control mice), FM−/− (FMOD knockout mice), Tyr−/− (albino mice) and Tyr−/−;FM−/− (albino and FMOD knockout mice). Tyr−/− have significantly more EC migration compared to the control mice and compared to the FM−/− mice. When the FMOD was knocked out in the Tyr−/− mice, the EC migration was comparable to that of the control mice and comparable to the FM−/− mice. This indicates that albino mice express significantly more FMOD and it is this excess FMOD that induces EC migration and promotes angiogenesis.

### Example 6

### Treatment of Experimental Choroidal Neovascularization (CNV) with Intravitreal Anti-fibromodulin

Experimental CNV was induced in normal C57B1 and albino mice C57-albino by laser as described previously. At the day of laser induction intravitreal injection of with anti-FMOD or any other fibromodulin activity inhibitor versus GST (control); a non-treated control experiment will also be performed. Fluorescence micrographs (Axio Observer Z1; Zeiss) on day 7 will be taken as perviously described, i.e., blood vessels in the retina will be stained with Alexa-594-isolectin and then imaged. Neovascular tuft formation will be quantified by comparing the number of pixels in the affected areas with the total number of pixels in the retina (Image J). Digitized images of the total retinal area and peripheral avascular areas will be measured, e.g., using the freeware ImageJ. The peripheral avascular area can be expressed as a percentage of the total retinal area. Neovascularization density will be quantified by summing capillary junctions within four equal areas, in each of the four quadrants of the vascularized retina determined by ImageJ. Areas of VO and NV will be quantified as percentages of total retina area. It is expected that the peripheral avascular areas will be greater in the treated mice compared to the control GST-treated and the non-treated control conditions. It is also expected that the neovascularization density will less in the anti-FMOD treated mice compared to the control GST-treated and the non-treated control conditions.

1. A method of treatment of age-related macular degeneration involving choroid neovascularization in the eye of a subject, the method comprising administering a therapeutically effective amount of fibromodulin activity inhibitor to the subject. having an eye color of 1-12 on the Martin-Schultz scale.

2. The method of claim 1, wherein the subject has an eye color of 1-12 on the Martin-Schultz scale.

3. The method of claim 1, further comprising selecting a subject diagnosed with having age-related macular degeneration.

4. The method of claim 1, wherein the fibromodulin activity inhibitor is selected from the group consisting of an antibody, an RNA interference molecule, a small molecule, a peptide and an aptamer.

5. The method of claim 1, wherein the fibromodulin activity inhibitor comprises an antibody.

6. The method of claim 1, wherein the subject is a mammal.

7. The method of claim 5, wherein the mammal is a human.

8. A method of inhibiting choroid neovascularization in the eye of a subject, the method comprising administering a therapeutically effective amount of a fibromodulin activity inhibitor to the subject.

9. The method of claim 8, wherein the subject has an eye color of 1-12 on the Martin-Schulz scale.

10. The method of claim 8, wherein the choroid neovascularization occurs in age-related macular degeneration.

11. The method of claim 8, wherein the fibromodulin activity inhibitor is selected from the group consisting of an antibody, an RNA interference molecule, a small molecule, a peptide and an aptamer.

12. The method of claim 8, wherein the fibromodulin activity inhibitor comprises an antibody.

13. A method of inhibiting fibromodulin activity in a subject comprising administering a therapeutically effective amount of a fibromodulin activity inhibitor to the subject.

14. The method of claim 13, wherein the subject has an eye color of 1-12 in the Martin-Schultz scale.

15. The method of claim 13, wherein the fibromodulin activity promotes choroid neovascularization.

16. The method of claim 13, wherein the fibromodulin activity occurs in age-related macular degeneration.

17. The method of claim 13, wherein the fibromodulin activity inhibitor is selected from the group consisting of an antibody, an RNA interference molecule, a small molecule, a peptide and an aptamer.

18. The method of claim 16, wherein the fibromodulin activity inhibitor comprises an antibody.

19. The method of claim 13, wherein the subject is a mammal.

20. The method of claim 18, wherein the mammal is a human.
